(19) United States
(12) Patent Application Publication
(10) Pub. No.: US 2024/0294869 A1
(43) Pub. Date: Sep. 5, 2024

(54) PIG XENOTRANSPLANTS INTO HUMANS WITHOUT CHRONIC IMMUNOSUPPRESSION

(71) Applicant: NUGIFT MEDICAL LLC, Greenwood, SC (US)

(72) Inventor: Kenneth Lawrence Franco, Greenwood, SC (US)

(21) Appl. No.: 18/178,282

(22) Filed: Mar. 3, 2023

(51) Int. Cl. C12N 5/071

(52) U.S. Cl. CPC C12N 5/0602; A01K 2207/15; A01K 2227/108

(57) ABSTRACT

Genetically modified animals suitable for use in xenotransplantation and methods of producing such animals suitable for use in xenotransplantation are provided. These animals and methods are particularly suited to human xenotransplantation. Specifically, the present application describes the production of genetically modified pigs lacking expression of glycoprotein alpha-galactosyltransferase 1 (GGTA1), cytidine monophospho-n-acetylneuraminic acid hydroxylase (CMAH), and beta-1,4-n-acetyl-galactosaminyltransferase 2 (B4GALNT2); and expressing human transgenes including human thrombomodulin (hTBM), human membrane cofactor protein (hCD46), and human decay accelerating factor for complement (hCD55). Preferably, the genetically modified pigs are Yucatan miniature pigs, and organs, tissues and cells derived therefrom are also provided. Method of inducing immune tolerance prior to the xenotransplantation by transferring the organ recipient's cells and factors into fetal pig donors and back to the organ recipient after the birth and development of the donor animal are also provided.

### FIELD OF THE INVENTION

The invention is generally directed to genetically modified donor animals, and organs, tissues, and cells thereof that are suitable for xenotransplantation therapies in humans.

### BACKGROUND OF THE INVENTION

There is a critical shortage of human organs for the purposes of organ transplantation. In the United States alone more than 100,000 patients are on waiting lists to receive organs, and yet only 30,000 or more organs will become available from deceased donors (Organ Procurement and Transplantation Network (OPTN)). 17 patients die each day waiting for an organ. The supply of human organs and tissues for use in allotransplantation will never fully meet the population's need.

Xenotransplantation (transplant of organs, tissues, and cells from a donor of a different species) could effectively address the shortage of human donor material. Xenotransplants are also advantageously (i) supplied on a predictable, non-emergency basis; (ii) produced in a controlled environment; and (iii) available for characterization and study prior to transplant.

Although xenotransplantation of organs, particularly from porcine donors, is an appealing alternative to the use of allografts because of the limited supply and quality of human donor materials, major obstacles remain. Both immediate and delayed immune responses require potentially toxic cocktails of immunosuppressant therapies, and even then, endothelial activation and subsequent coagulation dysregulation and thrombosis in the graft can cause graft failure. The production of genetically modified animals to address certain immune responses has been suggested; however, this requires the coordinated elimination or knockout of certain genes and appropriate expression or addition of certain transgenes that together are capable of addressing each immune response without significantly curtailing the overall health and viability of the pig. Thus, there remains a need for improved animals and tissues suitable for xenotransplantation therapies. In particular, there remains a need for improved donor animals, organs and tissues for use in xenotransplantation without requiring significant or long term immunosuppressive or anticoagulant therapies.

Therefore, it is an object of the invention to provide genetically modified porcine animals for xenotransplantation of vascularized xenografts and derivatives thereof.

It is another object to provide methods for providing vascularized xenografts and derivatives thereof for xenotransplantation without requiring significant or long term immunosuppressive or anticoagulant therapies.

### SUMMARY OF THE INVENTION

Genetically modified animals suitable for use in xenotransplantation and methods of producing animals suitable for use in xenotransplantation have been developed. Methods of producing and using genetically modified (GM) pig organs for xenotransplantation that are personalized to a specific organ recipient and that do not require chronic immunosuppression following xenotransplantation are provided. In particular, with the described donor animals and methods, when xenotransplant material is transplanted into a matched human recipient, the human recipient develops tolerance to the xenotransplant material because, due to the special techniques developed, the xenotransplant material will not be perceived as foreign. As a result, and for the first time in xenotransplantation, the human recipient will not need chronic immunosuppression.

The method conditions GM pig fetuses with specific cells and factors collected from the future organ recipient to make the pigs resulting from the treated fetuses more matched to the organ recipient, appearing more like self tissue to the organ recipient. This greatly reduces the anti-transplant reaction of the recipient and paves the way for xenotransplantation without the need for chronic immunosuppression. The method also conditions the future organ recipient with specific recipient's cells and factors conditioned and collected back from the recipient-matched pig (the future organ donor) to prepare the organ recipient's immune system prior to organ transplantation. This completes the reduction of the anti-transplant reaction of the recipient allowing xenotransplantation without the need for chronic immunosuppression.

The methods generally include the steps of (a) transferring cells (e.g., lymphocytes and stem cells) and factors of the organ recipient into a GM donor pig fetus in utero, (b) transferring, after completion of gestation of the GM donor pig fetus and delivery and some development of the resultant GM donor pig, cells and factors of the GM donor pig will be transferred back into the organ recipient, and (c) transplanting, 5 to 9 days following the transferring of cells and factors of the GM donor pig back into the organ recipient, an organ from the same GM donor pig is transplanted into the organ recipient.

Typically, the cells and factors of the organ recipient (i) are collected from blood and bone marrow of the organ recipient and (ii) are partially depleted of CD4+ and CD8+ T cells and the cells and factors of the GM donor pig (i) are collected from blood, bone marrow, spleen, cord blood and lymph nodes of the GM donor pig, (ii) are enriched for organ recipient cells and depleted of donor pig cells, (iii) are enriched for organ recipient foxP3+CD4+CD3+CD25+ natural T regulatory cells and expanded, (iv) are enriched for organ recipient adaptive CD4+CD49b+LAG3+CD226+ regulatory Tr1 cells and expanded, (v) are enriched for organ recipient CD4+CD25-CD69+foxP3-LAP+(TGFβ-producing type 3 helper T cells and expanded, and (vi) are enriched for organ recipient PD-L1 human dendritic cells and expanded.

The transferring of cells and factors of the organ recipient to the pig fetus is preferably accomplished by injecting the partially depleted cells and factors of the organ recipient into the abdominal cavity of the GM donor pig fetus. The donor pig fetus is preferably a genetically modified donor pig fetus being gestated in a genetically modified parent pig. The genetic modifications of the donor pig fetus are preferably the same as the genetic modifications of the parent pig. The genetic modifications are preferably limited to three gene knockouts and, optionally, three human transgenes. The GM donor pig fetus is preferably the same sex and compatible blood group as the organ recipient and the recipient preferably has low anti-pig antibodies (e.g., anti-SLA antibodies and anti-HLA antibodies). The organ recipient is preferably administered Cyclophosphamide and/or Fludarabine 5 to 9 days prior to the transferring the cells and factors of the GM donor pig back into the organ recipient.

Genetically engineered donor pigs useful for xenotransplantation of an organ that are personalized to a specific organ recipient and that do not require chronic immunosuppression following xenotransplantation are also provided. Typically, the donor pigs are genetically modified donor pigs that were delivered from a genetically modified parent pig. The genetic modifications of the donor pig are typically the same as the genetic modifications of the parent pig. Preferably, the genetic modifications are limited to three gene knockouts and, optionally, three human transgenes. The donor pig is preferably the same sex and blood type as the organ recipient and preferably has low anti-pig antibodies. The cells and factors of the organ recipient are transferred to the GM donor pig while the donor pig was a donor pig fetus in utero in the parent pig. The cells and factors of the organ recipient to be transferred are (i) collected from blood and bone marrow of the organ recipient and (ii) are partially depleted of CD4+ and CD8+ T cells.

Organs from GM donor pig that (i) are useful for xenotransplantation (ii) are personalized to a specific organ recipient, and (iii) do not require chronic immunosuppression following xenotransplantation are also described. Typically, the donor pig is a genetically modified donor pig that was delivered from a genetically modified parent pig. The genetic modifications of the donor pig are generally the same as the genetic modifications of the parent pig. The genetic modifications are preferably limited to three gene knockouts and, optionally, three human transgenes. The donor pig is generally the same sex and compatible blood group as the organ recipient and preferably has low anti-pig antibodies. The cells and factors of the organ recipient were ideally transferred to the GM donor pig while the donor pig was a donor pig fetus in utero in the parent pig. Prior to transferring, the cells of the organ recipient were preferably (i) collected from blood and bone marrow of the organ recipient and (ii) partially depleted of CD4+ and CD8+ T cells.

Cells and factors from a GM donor pig useful for supporting xenotransplantation of an organ of a donor pig that is personalized to a specific organ recipient and that does not require chronic immunosuppression following xenotransplantation are also provided. Typically, the donor pig is a genetically modified donor pig that was delivered from a genetically modified parent pig, where the genetic modifications of the donor pig are the same as the genetic modifications of the parent pig, where the genetic modifications are limited to three gene knockouts and, optionally, three human transgenes, and where the donor pig is the same sex and compatible blood group as the organ recipient and preferably has low anti-pig antibodies, where cells and factors of the organ recipient were transferred to the GM donor pig while the donor pig was a donor pig fetus in utero in the parent pig, and where, prior to transferring, the cells and factors of the organ recipient (i) were collected from blood and bone marrow of the organ recipient and (ii) were partially depleted of CD4+ and CD8+ T cells, (iii) where the cells and factors of the GM donor pig were collected from blood, bone marrow, cord blood, spleen, and lymph nodes of the donor pig, and/or (iv) were enriched for organ recipient cells and depleted of donor pig cells, and/or (v) were enriched for organ recipient foxP3+CD4+CD3+CD25+ natural T regulatory cells and expanded, and/or (vi) were enriched for organ recipient adaptive CD4+CD49b+LAG3+CD226+ regulatory Tr1 cells and expanded, and/or (vii) were enriched for organ recipient CD4+CD25-CD69+foxP3-LAP+(TGFβ-producing type 3 helper T cells) and expanded, and/or (viii) were enriched for organ recipient PD-L1 human dendritic cells and expanded.

A genetically modified pig is also described, where the genetic modifications are limited to three gene knockouts and, optionally, three human transgenes, where the gene knockouts are Glycoprotein Alpha-Galactosyltransferase 1 (GGTA1), Cytidine Monophospho-N-Acetylneuraminic Acid Hydroxylase (CMAH), and/or Beta-1,4-N-Acetyl-Galactosaminyltransferase 2 (B4GALNT2), where the human transgenes are human Thrombomodulin (hTBM), human Membrane Cofactor Protein (hCD46), and human Decay Accelerating Factor For Complement (hCD55).

In preferred forms, the methods will use small Yucatan pigs to make genetically-modified (GM) pig so that the pig organs will not continue to grow in the recipient after xenotransplantation, as outlined in FIG. 1.

In preferred forms, the pig population will be expanded with minimal in-breeding to meet the large demand for organs required.

In preferred forms, the genetic modifications to the donor pigs will be limited so as not to weaken the pig genome and to allow the pig organs to function for many years in the recipient.

The genetic modifications can be made using any suitable technique, such as somatic cell nuclear transfer (SCNT) or CRISPR-Cas. In preferred forms, the genetic modifications will be made using somatic cell nuclear transfer (SCNT) and additional techniques and/or other methods so as to avoid any potential problems with long term pig organ function.

In preferred forms, the GMs in the pig include 6 modifications (knockout 3 pig genes, add three human trans genes).

In preferred forms, Porcine Endogenous Retrovirus-C (PERV-C) will be eliminated in the pig organ.

In preferred forms, the GM pigs will be raised in pathogen free environments, tested for all infectious agents and eliminate all viruses of concern.

In preferred forms, recipients will have their own matched GM pig, with the pig organs being repopulated with their own cells to make more humanized pig organs.

In preferred forms, the removal of recipient lymphocytes will be standardized, with the removal of stem cells (hematopoietic and mesenchymal) and other factors from the recipient's blood and bone marrow.

In preferred forms, the injection of the recipient's lymphocytes will be standardized, with the injection of stem cells and factors into the donor pigs in-utero (track the cells).

In preferred forms, the recipient's lymphocytes and stems cell will undergo differentiation and maturation into regulatory cells, suppressor cells, select B cells, and antibodies in the pig donor while in utero. Another potential benefit of infusing human lymphocytes, stem cells and factors into the fetal pig is the potential for accommodation of the pig endothelial cells to human proteins which may reduce the potential metabolic incompatibilities associated with protein/ligand differences between species. After the pig is born these antigen specific regulatory cells, etc. will be responsible for providing specific tolerance when these cells are given back to the recipient in conjunction with the pig organ from the same pig.

In preferred forms, the removal, expansion, and purification of cells from the donor piglet after delivery will be standardized.

In preferred forms, the removal, expansion, and purification of human cells after the pig fetus is delivered will be standardized, with the cells including: (i) foxP3+CD4+CD3+CD25+ natural T regulatory cells, (ii) adaptive CD4+CD49b+LAG3+CD226+ regulatory Tr1 cells, (iii) CD4+CD25-CD69+foxp3-LAP+(TGF beta producing type 3 helper T cells (TH 3), and (iv) dendritic cells expressing PD-L1.

In preferred forms, the injection of the regulatory T cells and the dendritic cells back into the recipient who originally donated the lymphocytes and stem cells to the donor pig fetus will be standardized.

In preferred forms, the methods include the combination of steps of (a) transferring cells (e.g., lymphocytes and stem cells) and factors of the organ recipient into a GM donor pig fetus in utero, where, prior to transferring, the cells and factors of the organ recipient (i) are collected from blood and bone marrow of the organ recipient and (ii) are partially depleted of CD4+ and CD8+ T cells, where the transferring is accomplished by injecting the partially depleted cells and factors of the organ recipient into the abdominal cavity of the GM donor pig fetus, where the donor pig fetus is a genetically modified donor pig fetus being gestated in a genetically modified parent pig, where the genetic modifications of the donor pig fetus are the same as the genetic modifications of the parent pig, where the genetic modifications are limited to three gene knockouts and, optionally, three human transgenes, and where the GM donor pig fetus is the same sex and compatible blood group as the organ recipient and preferably has low anti-pig antibodies; and (b) transferring, after completion of gestation of the GM donor pig fetus and delivery and some development of the resultant GM donor pig, cells and factors of the GM donor pig into the organ recipient, where, prior to transferring, the cells and factors of the GM donor pig (i) are collected from blood, bone marrow, spleen, cord blood and lymph nodes of the GM donor pig, (ii) are enriched for organ recipient cells and depleted of donor pig cells, (iii) are enriched for organ recipient foxP3+CD4+CD3+CD25+ natural T regulatory cells and expanded, (iv) are enriched for organ recipient adaptive CD4+CD49b+LAG3+CD226+ regulatory Tr1 cells and expanded, (v) are enriched for organ recipient CD4+CD25-CD69+foxP3-LAP+(TGFβ-producing type 3 helper T cells) and expanded, and (vi) are enriched for organ recipient PD-L1 human dendritic cells and expanded, where, 5 to 9 days prior to transferring, the organ recipient is administered Cyclophosphamide and/or Fludarabine and (c) transplanting, 5 to 9 days following the transferring of cells and factors of the GM donor pig into the organ recipient, an organ from the same GM donor pig is transplanted into the organ recipient.

### BRIEF DESCRIPTION OF THE DRAWING

FIG. 1 is a diagram of Yucatan weight-age chart showing the pig weight varies no more than one standard deviation away from the average over time.

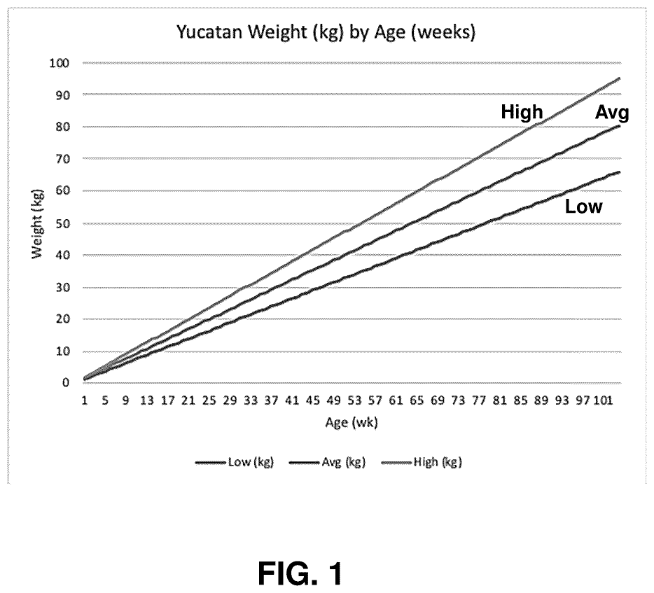

FIG. 2 is a schematic diagram showing the exemplary method of generating genetically modified pigs including GGTA−/−, CMAH−/−, B4GALNT2−/−, Xeno 3 pigs and GGTA−/−, CMAH−/−, B4GALNT2−/−, hTBM/hCD46/hCD55 Xeno 6 pigs.

FIG. 3 is a schematic diagram showing the transfer of cells and factors from an organ recipient to a fetal donor pig, where the fetal donor pig later serves as a source of both tolerance-inducing immunosuppressive cells and factors and a transplant organ.

### DETAILED DESCRIPTION OF THE INVENTION

A method has been developed to produce genetically modified, specially treated mini-donor pigs with cells and factors of a specific human recipient. Organs of this mini-donor pig can be used for xenotransplantation. Prior to organ transplantation, specific human cells and factors from this mini-donor pig are transferred back to the human recipient. After the mini-pig organ is transplanted into the human recipient, the recipient develops tolerance to the pig organ because, due to the techniques developed, the organ will not be perceived as foreign. As a result, and for the first time, the recipient will not need chronic immunosuppression. No prior method has produced such results.

Conceptionally, the preferred forms of the method involve the following basic steps:
1. Special mini-pigs will be used, which will not allow the organs to continue to grow after the organ is transplanted into a human recipient.
2. Limited genetic modifications in the mini-pig so as not to weaken the pig genome but prevent hyperacute and delayed vascular xeno-rejection of the pig donor organ after it is transplanted into a human (knockout genes and insert human transgenes).
3. Special genetically modified mini-pigs raised in pathogen free environments and tested for all infectious agents of concern.
4. Specific cell therapy for both the donor mini-pig and human recipient, which will allow the human recipient to develop tolerance to the transplanted pig organ and not need chronic immunosuppression.
   a. Standardized removal of specific cells (lymphoctyes and stem cells) and factors from the human recipient and inject them into donor mini-pig.
   b. Standardized removal of specific human cells (lymphocytes and stem cells) and factors from the donor mini-pig and expand them in vitro and inject them back into the human recipient.
   c. Transplant the pig organ from same donor mini-pig containing specific human cells and factors into the human recipient who originally supplied the specific human cells and factors.
5. No chronic immunosuppression.
6. Anti-inflammatory drug post-xenotransplantation.

Genetically modified animals suitable for use in xenotransplantation and methods of producing such animals suitable for use in xenotransplantation are provided. These animals and methods are particularly suited to human xenotransplantation. Specifically, the present application describes the production of genetically modified pigs lacking expression of glycoprotein alpha-galactosyltransferase 1 (GGTA1), cytidine monophospho-n-acetylneuraminic acid hydroxylase (CMAH), and beta-1,4-n-acetyl-galactosaminyltransferase 2 (B4GALNT2); and expressing human transgenes including human thrombomodulin (hTBM), human membrane cofactor protein (hCD46), and human decay accelerating factor for complement (hCD55). Preferably, the genetically modified pigs are Yucatan miniature pigs, and organs, tissues and cells derived therefrom are also provided. Method of inducing immune tolerance prior to the xenotransplant by transferring the organ recipient's cells and factors into fetal pig donors and back to the organ recipient after the birth and development of the donor animal are also provided.

### I. Definitions

An organ graft is herein defined to mean a solid organ, tissue, or cells to be transplanted.

An organ graft recipient is defined herein to mean an animal such as a human intended to be the final recipient of an organ graft.

An organ donor is defined herein to mean a donor animal intended to have organ recipient's hematopoietic stem cells, mesenchymal stem cells, lymphocytes and factors develop within the donor animal. The donors are similar to chimeras, i.e., animals engrafted or infused with the recipient's cells. These cells and resulting factors differentiate and mature into regulatory cells, suppressor cells, select B cells, and antibodies, so when transferred back into the recipient, the recipient will develop immune tolerance to the tissues of the donor animal. Another potential benefit of infusing human cells and factors into the fetal pig is the potential for accommodation of the pig endothelial cells to human proteins which may reduce the potential metabolic incompatibilities associated with protein/ligand differences between species. The same donor preferably also provides the organ graft. The donor is always allogeneic, meaning genetically non-identical to the recipient, and usually xenogeneic, meaning of a species different from the recipient.

The term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, Reverse Transcription Polymerase Chain Reaction (RT-PCR), in situ hybridization, Western blotting, and immunostaining such as Fluorescence Activated Cell Sorting (FACS). In some forms, expression of one or more genes is introduced (e.g., transgenes) in the genetically modified pigs such as human thrombomodulin, human CD46, and human CD55.

The term “culturing . . . with” is intended to include incubating the component(s) and the cell/tissue together in vitro (e.g., adding the compound to cells in culture) and the step of “culturing . . . with” can be conducted in any suitable manner. For example, the cells may be treated in adherent culture, in suspension culture, or in 3D culture; the components can be added temporally, substantially simultaneously (e.g., together in a cocktail) or sequentially (e.g., within 1 hour, 1 day or more from an addition of a first component). The cells can also be contacted with another agent such as a growth factor or other differentiation agent or environments to stabilize the cells, or to differentiate the cells further and include culturing the cells under conditions known in the art. In some forms, cells are cultured to expand for a more desirable number of cells before transferring to a donor or recipient.

The terms “inhibit” or “reduce” generally mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%, or an integer there between. In some forms, the inhibition and reduction are compared at mRNAs, proteins, cells, tissues, and organs levels. In some forms, expression of one or more genes is reduced or eliminated (e.g., knockout).

The terms “prevent”, “prevention” or “preventing” mean to administer a composition or method to a subject or a system at risk for or having a predisposition for one or more symptom caused by transplant rejection, to decrease the likelihood the subject will develop one or more symptoms of the transplant rejection, or to reduce the severity, duration, or time of onset of one or more symptoms of the transplant rejection.

### II. Donor Animals with Genetic Modification

While advantageous in many ways, xenotransplantation also creates a more complex immunological scenario than allotransplantation. As such, considerable effort has been directed at addressing the immune barriers through genetic modification (van der Windt et al., Xenotransplantation. 2007 July; 14(4):288-97, Cowan and D'Apice, Curr Opin Organ Transplant. 2008 April; 13(2):178-83). Xenograft rejection can be divided into three phases: hyperacute rejection (HAR), acute humoral xenograft rejection (AHXR), and T cell-mediated cellular rejection.

HAR is a very rapid event that results in irreversible graft damage and loss within minutes to hours following graft reperfusion. HAR is defined by the ubiquitous presence of high titers of pre-formed natural antibodies present within the recipient at the time of transplantation. The binding of these natural antibodies to target epitopes on the donor tissue endothelium is believed to be the initiating event in HAR. This binding, within minutes of perfusion of the donor tissue with the recipient blood, is followed by complement activation, platelet, and fibrin deposition, and ultimately by interstitial edema and hemorrhage in the donor organ, all of which cause rejection of the tissue in the recipient (Strahan et al. (1996) Frontiers in Bioscience 1, e34-41). The primary cause of HAR in humans is the natural anti-Gal antibody, which comprises approximately 1% of antibodies in humans and monkeys. This initial hyperacute rejection is then reinforced by the delayed vascular response (also known as acute humoral xenograft rejection (AHXR), acute vascular xeno-rejection (AVXR) or delayed xenograft rejection (DXR)). The lysis and death of endothelial cells during the hyperacute response is accompanied by edema and the exposure of adventitial cells, which constitutively express tissue factor (TF) on their surface. Tissue factor is thought to be pivotal in the initiation of the in vivo coagulation cascade, and its exposure to plasma triggers the clotting reactions. Thrombin and TNF-alpha become localized around the damaged tissue, and this induces further synthesis and expression of TF by endothelial cells. The environment around resting endothelial cells does not favor coagulation. Several natural coagulation inhibitors are associated with the extracellular proteoglycans of endothelial cells, such as tissue factor pathway inhibitor, antithrombin III, and thrombomodulin. The recognition of the foreign tissue by xeno-reactive natural antibodies (XNAs), however, causes the loss of these molecules. Together with the exposure and induction of tissue factor, the anticoagulant environment around endothelial cells thus becomes pro-coagulant. The vascularized regions of the xenograft thus become sites of blood clots, a characteristic of damaged tissue. Blood flow is impaired, and the transplanted organ becomes ischemic.

Humans have a naturally occurring antibody to the alpha 1,3 galactose (Gal) epitope found on pig cells. This antibody is produced in high quantity and, it is now believed, is the principle mediator of HAR. Initial efforts to genetically modify pigs have focused on removing the alpha 1,3-galactose (Gal) epitope from pig cells. In 2003, Phelps et al. (Phelps et al., Science, 2003, 299:411-414) reported the production of the first live pigs lacking any functional expression of aGT (GTKO), which represented a major breakthrough in xenotransplantation (see also PCT publication No. WO 04/028243 to Revivicor, Inc. and PCT Publication No. WO 04/016742 to Immerge Biotherapeutics, Inc.). Subsequent studies have shown that organ grafts from GTKO pigs do not undergo HAR (Kuwaki et al., Nat Med. 2005 January; 11(1):29-31, Yamada et al., Nat Med. 2005 January; 11(1):32-4).

Genetically modified donor animals that provide organs, tissues and cells that are particularly useful for xenotransplantation therapies in human recipients are described. The genetically modified donor animals serve as a source of organs, tissues and cells that overcome significant humoral and cellular immune responses, making them useful for xenotransplantation.

Production of genetically modified animals requires the coordinated elimination or knockout of donor genes and appropriate expression or addition of multiple transgenes that can address each immune response without significantly curtailing the overall health and viability of the donor animals. Specifically, the genetically modified donor pigs are discussed in detail below.

In some forms, the donor pigs are genetically modified to reduce or remove the expression of one or more genes that express antigens that can be recognized by naturally occurring antibodies in human. Exemplary gene knockouts include glycoprotein alpha-galactosyltransferase 1 (GGTA1), cytidine monophospho-n-acetylneuraminic acid hydroxylase (CMAH), and beta-1,4-n-acetyl-galactosaminyltransferase 2 (B4GALNT2); and the organs, tissues and cells derived therefrom are also provided.

In some forms, expression or addition of transgenes critical to overcome transplant rejection is desirable, for example genes encoding one or more of anticoagulants, immunomodulators and cytoprotectants. In some forms, the donor pigs are genetically modified to express one or more human transgenes. Exemplary human transgenes include human thrombomodulin (hTBM), human membrane cofactor protein (hCD46), and human decay accelerating factor for complement (hCD55); and the organs, tissues and cells derived therefrom are also provided.

In one form, donor pigs are Yucatan miniature pigs genetically modified to eliminate or knockout the expression of GGTA1, CMAH, and B4GALNT2; and to express three human transgenes of hTBM, hCD46, and hCD55; and organs, tissues and cells derived therefrom, are also provided.

Once transgenic animals are produced, tissues, including skin, heart, livers, kidneys, lungs, pancreas, small bowel, eyes (e.g., retina and cornea), and components thereof are harvested and can be implanted as known by those skilled in the art of transplantation.

### A. Gene Knockout

In some forms, the donor pigs are genetically modified to reduce or remove the expression of one or more genes that express antigens that can be recognized by naturally occurring antibodies in human. Exemplary gene knockouts include glycoprotein alpha-galactosyltransferase 1 (GGTA1), cytidine monophospho-n-acetylneuraminic acid hydroxylase (CMAH), and beta-1,4-n-acetyl-galactosaminyltransferase 2 (B4GALNT2). In some forms, the genetically modified pigs having one or more gene knockouts of GGTA1, CMAH, and B4GALNT2, as well as the organs, tissues and cells derived therefrom are provided. In preferred forms, genetically modified pigs are Yucatan miniature pigs having three gene knockouts including GGTA1, CMAH, and B4GALNT2, as well as the organs, tissues and cells derived therefrom, are provided.

### 1. Glycoprotein Alpha-Galactosyltransferase 1 (GGTA1)

The alpha 1,3 galactosyltransferase (GGTA1) gene encodes an enzyme (GT, α1,3 galactosyltransferase). Ensemble transcript ENSSSCG00000005518 includes the porcine GGTA1 nucleotide sequence. Functional α1,3 galactosyltransferase catalyzes formation of galactose-α1,3-galactose residues on glycoproteins. The galactose-α1,3-galactose (αGa1) residue is an antigenic epitope or antigen recognized by the human immunological system.

To prevent expression of the α 1->3 galactosyl transferase, the gene may be deleted, interrupted, or replaced, either within the coding region or within the regulatory sequences, so that enzyme is not produced. This is generally accomplished by manipulation of animal embryos followed by implantation of the embryos in a surrogate mother. The embryos can be manipulated directly, by injection of genetic material into the embryo by microinjection or by vectors such as retroviral vectors, or indirectly, by manipulation of embryonic stem cells. The latter methodology is particularly useful in the case where the end result that is desired is to completely prevent expression of an active enzyme.

### 2. Cytidine Monophospho-N-Acetylneuraminic Acid Hydroxylase (CMAH)

Another earliest known xenoantigens other than gal-α-gal is an epitope that Hanganutziu Deicher antibodies recognize, and which have long been associated with serum disease (Hanganutziu, M., CR Soc. Biol. (Paris), 91, p. 1457 (1924); Deicher, H., Z. Hyg., 106, p. 561 (1926)). The epitope has been identified as N-glycolylneuraminic acid (Neu5Gc), a member of the sialic acid family of carbohydrates. Among carbohydrates, sialic acids are abundant and ubiquitous. Sialic acid is a generic designation used for N-acylneuraminic acids (Neu5Acyl) and their derivatives. N-Acetylneuraminic acid (Neu5Ac) and N-glycolylneuraminic acid (Neu5Gc) are two of the most abundant derivatives of sialic acids. The Neu5Gc epitope is located in the terminal position in the glycan chains of glycoconjugates. Due to this exposed position, it plays an important role in cellular recognition, e.g., in the case of inflammatory reactions, maturation of immune cells, differentiation processes, hormone-, pathogen- and toxin binding.

Glycoconjugates containing Neu5Gc are immunogenic in humans. In healthy humans, Neu5Gc is not detectable, although Neu5Gc is abundant in most mammals. The lack of Neu5Gc in man is due to an exon deletion in the human gene that prevents the formation of functional enzyme (Chou, H. H., et al. Proc. Natl. Acad. Sci. (USA), 95, pp. 11751-11756 (1998); Irie, A., et al. J. Biol. Chem., 273, pp. 15866-15871 (1998)). Thus, Neu5Gc-containing glycoconjugates act as antigens and can induce the formation of antibodies.

The Neu5Gc epitope is formed by the addition of a hydroxyl group to the N-acetyl moiety of Neu5Ac. The enzyme that catalyzes the hydroxylation is cytidine monophosphate-N-acetylneuraminic acid hydroxylase (CMP-Neu5Ac hydroxylase gene, CMAH). The Ensembl database id Gene: ENSSSCG00000001099 includes the porcine CMAH nucleotide sequence. Thus, the expression of the CMP-Neu5Ac hydroxylase gene determines the presence of the Neu5Gc epitope on cell surfaces.

In some forms, the CMAH is deleted, interrupted, or replaced, either within the coding region or within the regulatory sequences, so that CMAH is not produced. This is generally accomplished by manipulation of animal embryos followed by implantation of the embryos in a surrogate mother. The embryos can be manipulated directly, by injection of genetic material into the embryo by microinjection or by vectors such as retroviral vectors, or indirectly, by manipulation of embryonic stem cells. The latter methodology is particularly useful in the case where the end result that is desired is to completely prevent expression of an active enzyme.

### 3. Beta-1,4-N-Acetyl-Galactosaminyltransferase 2 (B4GALNT2)

The porcine beta-1,4-N-acetyl-galactosaminyltransferase 2 (B4GALNT2) enzyme is homologous to the human enzyme, which synthesizes the human SDa blood group antigen. Most humans produce low levels of anti-SDa IgM which polyagglutinates red blood cells from rare individuals with high levels of SDa expression. The SDa glycan is also present on GM2 gangliosides. Clinical GM2 vaccination studies for melanoma patients suggest that a human antibody response to SDa can be induced. Expression of porcine B4GALNT2 in human HEK293 cells results in increased binding of anti-SDa antibody and increased binding of Dolichos biflorus agglutinin (DBA), a lectin commonly used to detect SDa. In pigs, B4GALNT2 is expressed by vascular endothelial cells and endothelial cells from a wide variety of pig backgrounds stain with DBA, suggesting that porcine vascular expression of B4GALNT2 is not polymorphic. Mutations in B4GALNT2 have been engineered in mice and pigs. In both species, the B4GALNT2-KO animals are apparently normal and no longer show evidence of SDa antigen expression. Pig tissues with a mutation in B4GALNT2, added to a background of alpha-1,3-galactosyltransferase deficient (GGTA1-KO) and cytidine monophosphate-N-acetylneuraminic acid hydroxylase deficient (CMAH-KO), show reduced antibody binding, confirming the presence of B4GALNT2-dependent antibodies in both humans and non-human primates (Byrne G et al., Xenotransplantation. 2018 September; 25(5):e12394). Elimination of this source of immunogenic pig antigen should minimize acute injury by preformed anti-pig antibody and eliminate an induced clinical immune response to this xenotransplantation antigen.

In some forms, the B4GALNT2 is deleted, interrupted, or replaced, either within the coding region or within the regulatory sequences, so that B4GALNT2 is not produced. This is generally accomplished by manipulation of animal embryos followed by implantation of the embryos in a surrogate mother. The embryos can be manipulated directly, by injection of genetic material into the embryo by microinjection or by vectors such as retroviral vectors, or indirectly, by manipulation of embryonic stem cells. The latter methodology is particularly useful in the case where the end result that is desired is to completely prevent expression of an active enzyme.

In preferred forms, donor pigs are genetically modified to eliminate the expression of GGTA1, CMAH, and B4GALNT2. The organs, tissues and cells derived therefrom are also provided.

### B. Expression of Human Transgenes

In some forms, expression of transgenes critical to overcome transplant rejection is desirable, for example genes encoding one or more of anticoagulants, immunomodulators and cytoprotectants. In some forms, the donor pigs are genetically modified to express one or more human transgenes. Exemplary human transgenes include human thrombomodulin (hTBM), human membrane cofactor protein (hCD46), and human decay accelerating factor for complement (hCD55).

In some forms, the genetically engineered pigs lacking the expression of GGTA1, CMAH, and B4GALNT2 are further modified to express one or more human transgenes such as thrombomodulin, CD46, and CD55. In preferred embodiments, the genetically engineered pigs lacking the expression of GGTA1, CMAH, and B4GALNT2 are further modified to express all three human transgenes including thrombomodulin, CD46, and CD55 to make these animals suitable donors for xenotransplantation of vascularized xenografts and derivatives thereof. Organs, tissues and cells derived therefrom are also provided.

1. Human Thrombomodulin (hTBM)

In some forms, the donor pigs are modified to express human thrombomodulin.

Xenotransplantation is associated with an inflammatory response. Thrombomodulin (TBM), which plays a role in maintaining an anticoagulant state, also has an anti-inflammatory effect through both coagulation-dependent and coagulation-independent pathways (Hagiwara S et al., Shock. 2010; 33: 282-288).

2. Human Membrane Cofactor Protein (hCD46)

Complement is the collective term for a series of blood proteins and is a major effector mechanism of the immune system. Complement activation and its deposition on target structures can lead to direct complement-mediated cell lysis or can lead indirectly to cell or tissue destruction due to the generation of powerful modulators of inflammation and the recruitment and activation of immune effector cells. Complement activation products that mediate tissue injury are generated at various points in the complement pathway. Inappropriate complement activation on host tissue plays an important role in the pathology of many autoimmune and inflammatory diseases and is also responsible for many disease states associated with bio-incompatibility, e.g., post-cardiopulmonary inflammation and transplant rejection. Complement deposition on host cell membranes is prevented by complement inhibitory proteins expressed at the cell surface. The complement system comprises a collection of about 30 proteins and is one of the major effector mechanisms of the immune system. The complement cascade is activated principally via either the classical (usually antibody-dependent) or alternative (usually antibody-independent) pathways.

Membrane inhibitors of complement activation include complement receptor 1 (CR1), decay-accelerating factor (DAF or CD55) and membrane cofactor protein (MCP or CD46). Transgenic pigs expressing decay acceleration factor DAF (CD55), membrane co-factor protein MCP (CD46) and membrane inhibitor of reactive lysis, MIRL (CD59) have been generated (Klymium et al. Mol Reprod Dev (2010)77:209-221). These human inhibitors have been shown to be abundantly expressed on porcine vascular endothelium. Ex vivo perfusion of hearts from control animals with human blood caused complement-mediated destruction of the organ within minutes, whereas hearts obtained from transgenic animals were refractory to complement and survived for hours.

CD46 has been characterized as a protein with regulatory properties able to protect the host cell against complement mediated attacks activated via both classical and alternative pathways (Barilla-LaBarca, M. L. et al., J. Immunol. 168, 6298-6304 (2002)). hCD46 may offer protection against complement lysis during inflammation and humoral rejection mediated by low levels of natural or induced anti-Gal or anti-nonGal antibodies. Transgenic pigs with the combination of GTKO and expression of CD46 provided prolonged survival and function of xenograft hearts (pig-to baboon) for up to 8 months without any evidence of immune rejection (Mohiuddin et al., Abstract TTS-1383. Transplantation 2010; 90 (suppl): 325).

Accordingly, in some forms, the donor pigs are modified to express human CD46. In preferred forms, the genetically engineered pigs lacking the expression of GGTA1, CMAH, and B4GALNT2 are further modified to express human CD46.

3. Human Decay Accelerating Factor for Complement (hCD55)

The human decay-accelerating factor (DAF), encoded by the CD55 gene, is mostly referred to as a membrane-bound glycoprotein anchoring via phosphatidylinositol. DAF's ability to recognize and sequester C4b and C3b fragments, created either during the classical or alternative complement activation, prevents the amplification of the complement cascade, thus effectively downplaying the establishment of the membrane attack complex (Ward T., et al., EMBO J. 1994; 13:5070-5074; Ozen A., Comrie W. A., et al., N. Engl. J. Med. 2017; 377:52-61). Extended survival times have been observed for organs transplanted from human CD55-transgenic pigs to primates (Byrne G W, et al., (1997) Transplantation 63, 149-155). Studies have shown that hCD55 transgenic pigs have decreased susceptibility to HAR (Martínez-Alarcón L, et al., Biology (Basel). 2021 August; 10(8): 747).

Accordingly, in some forms, the donor pigs are modified to express human CD55. In preferred forms, the genetically engineered pigs lacking the expression of GGTA1, CMAH, and B4GALNT2 are further modified to express human CD55.

### C. Xenograft Material

Xenografts derived from a GM animal are also described. Typically, the xenograft material includes tissues, organs, and bodily substances to be transplanted.

As defined herein, a “xenograft” may be an organ or tissue (or aggregates of cells, or cells, collectively referred to herein as “tissue”). The tissue may be selected from any appropriate tissue of the body of the tissue donor. The term “xenotransplantation”, as used herein, refers to a procedure that involves the transplantation, implantation, or infusion into a recipient subject either (a) live cells, tissues, or organs from a nonhuman animal source, or (b) human body fluids, cells, tissues or organs that have had ex vivo contact with live nonhuman animal cells, tissues or organs.

Exemplary xenografts include, but are not limited to, heart, kidneys, lungs, pancreatic islet cells, liver, skin and/or epithelium, pancreas, intestine (e.g., small bowel), blood vessels (e.g., aorta), eyes (e.g., retina and cornea), and components thereof.

In some forms, the xenograft is a hollow or tubular organ for the transport of anabolites and catabolites, that is, vessels, trachea, and esophagus, ureter and urethra, and intestine.

In particular forms, the xenograft is all or part of the heart. Exemplary components of the heart that can form all or part of the xenograft include the superior vena cava, pulmonary vein, right atrium, pulmonary valve, tricuspid valve, inferior vena cava, aorta, pulmonary artery, left atrium, mitral valve, aortic valve and left ventricle.

In some forms, the xenograft is or includes all or part of the pancreas and/or islet cells.

In some forms, the xenograft material is one organ or tissue type. In other forms, the xenograft material includes more than one organ or tissue type. For example, in some forms, the xenograft material includes multiple organs and/or tissue types. Typically, the xenograft is or includes live nonhuman animal cells. In some forms, the xenograft further includes a bodily fluid. In particular forms, the xenograft includes blood, or one or more fractions thereof, such as plasma, white blood cells and/or red blood cells. In certain forms, blood or one or more fractions thereof is the only xenograft material that is obtained and/or transplanted.

When the xenograft is or includes nonhuman animal cells, the cells can be differentiated cells, or non-differentiated cells, such as stem cells. In some forms, the xenograft is or includes neuronal cells, islet cells, adrenal chromaffin cells, bone marrow, bone cells, tendon, cartilage, hepatocytes, myocardial cells or renal cells. In some forms, the xenograft is or includes nonhuman animal stem cells. In some forms, the xenograft is or includes nonhuman animal embryonic cells, such as fetal neuronal cells. Therefore, in some forms, the xenograft includes one or more cells types including skin and bone cells (e.g., fibroblasts, osteoblasts, osteocytes, osteoclasts, osteoprogenitor cells), cardiac cells (e.g., cardiac fibroblasts (CFs) and cardiomyocytes, smooth muscle cells (SMCs), endothelial cells (ECs)), neural cells (e.g., neurons, glial cells, astrocytes, oligodendrocytes, ependymal cells, and microglia, and choroid plexus cells), blood cells (e.g., red blood cells, white blood cells), liver cells (e.g., hepatocytes (HCs), hepatic stellate cells (HSCs), Kupffer cells (KCs), and liver sinusoidal endothelial cells (LSECs)), kidney cells (e.g., glomerular basement membrane/glomerular endothelial cells, macula densa cells, mesangial cells, podocytes, tubule epithelial cells), pancreatic cells (e.g., islet cells, including alpha, beta and delta cells), and stem cells.

In some forms, the xenograft is or includes nonhuman animal cells and tissues that are necessary and sufficient for repair or replacement of human tissue that is diseased or damaged. For example, in some forms, the xenograft includes skin and/or epithelium and/or hair that is required to repair skin that is damaged, for example, due to trauma such as a burn, or diseased, such as by cancer. Therefore, in some form, the xenograft is in an amount effective to treat an injury or disease in a recipient subject.

In some forms, the xenograft is or includes a bioartificial device, such as a bioartificial organ. Bioartificial organs implement the design, modification, growth and maintenance of living tissues embedded in natural or synthetic scaffolds to enable them to perform complex biochemical functions, including adaptive control and the replacement of normal living tissues. Therefore, in some forms, the xenograft is a bioartificial device that includes non-human animal cells and a prosthesis. In some forms, the bioartificial device is a bioartificial kidney device, bioartificial liver device, or a bioartificial heart device.

### III. Methods of Generating Genetically Modified Donor Animals

Methods of producing animals suitable for use in xenotransplantation are provided. In some forms, the methods produce genetically modified pigs lacking expression of glycoprotein alpha-galactosyltransferase 1 (GGTA1), cytidine monophospho-n-acetylneuraminic acid hydroxylase (CMAH), and beta-1,4-n-acetyl-galactosaminyltransferase 2 (B4GALNT2); and expressing human transgenes including human thrombomodulin (hTBM), human membrane cofactor protein (hCD46), and human decay accelerating factor for complement (hCD55).

Donor animals are provided. The donor animal is a non-human mammal, such as a pig. Typically, the animal is of an appropriate size such that organs and tissues that are to be transplanted from the animal can readily be implanted into a human recipient without significant changes in the size and/or shape of the organs and tissue.

In some forms, the donor animal is a pig. Exemplary pig species include Sus scrofa domesticas, Sus ahoenobarbus, Sus barbatus, Sus bucculentus, Sus cebifrons, Sus celebensis, Sus heureni, Sus oliveri, Sus philippensis, and Sus verrucosus. In some forms, the pig is a breed of domestic pig (i.e., a Sus scrofa domesticas pig). An exemplary domestic pig is a Yucatan pig.

Pigs have relatively short gestation periods, and thus can be bred to meet the large demand for organs required. In some forms, the genetically modified donor pigs are bred in pathogen-free environments. In some forms, the genetically modified donor pigs are tested for most infectious agents and those of concern especially those carrying viruses are eliminated. The disclosed methods make all genetic modifications in the donor pig and none in the recipient. Each human recipient will have their own genetically modified donor pig and organs/tissues/cells with which the recipient's own cells have been conditioned prior to being transferred back to the recipient and prior to xenotransplant of the pig organs/tissues/cells.

### A. Selection of Pigs

Pigs have been the focus of most research in the xenotransplantation area, since the pig shares many anatomical and physiological characteristics with human. Pigs also have relatively short gestation periods, can be bred in pathogen-free environments, and may not present the same ethical issues associated with animals not commonly used as food sources (e.g., primates). Scientific knowledge and expertise in the field of pig-to-primate xenotransplantation has grown rapidly over the last decade, resulting in the considerably prolonged survival of primate recipients of lifesaving porcine xenografts. (Cozzi et al., Xenotransplantation, 16:203-214. 2009). Pigs can include any pig known to the art including, but not limited to, a wild pig, domestic pig, mini pigs, a Sus scrofa pig, a Sus scrofa domesticus pig, as well as in-bred pigs. Pigs can be selected from Landrace, Yorkshire, Hampshire, Duroc, Chinese Meishan, Chester White, Berkshire Goettingen, Landrace/York/Chester White, Yucatan, Bama Xiang Zhu, Wuzhishan, Xi Shuang Banna and Pietrain pigs.

Minipigs play an important role in biomedical research and they have also been used as donor animals for preclinical xenotransplantations. Since zoonotic microorganisms including viruses can be transmitted when pig cells, tissues or organs are transplanted, virus safety is an important feature in xenotransplantation. Whereas most porcine viruses can be eliminated from pig herds by different strategies, this is also possible for porcine endogenous retroviruses (PERVs). PERVs are integrated in the genome of pigs and some of them release infectious particles able to infect human cells. To date, PERV viruses have not been transmitted into humans, PERV-C appears to be more of a concern and if not eliminated by genetic means and should it cause an infection in humans it can be treated with anti-retroviral drugs. In preferred forms, the disclosed methods use pigs that are negative for PERV-C.

The most known minipigs are the Minnesota minipigs from the Hormel Institute in the United States, the Massachusetts General Hospital swine leukocyte antigen (SLA)-defined miniature pigs, the Göttingen minipigs, the Aachen minipigs, the Munich miniature swine, the Yucatan miniature pigs, the Chinese bama minipigs, the Brazilian minipig and the Mini-LEWE. The Minnesota miniature swine were the first minipigs developed in the USA in 1940s and they are the basis of most if not all other minipig breeds, including the Göttingen minipigs, which were the first miniature pig breed and developed in Europe (Denner J. and Jan Schuurman H., Viruses. 2021 September; 13(9): 1869).

The Yucatan miniature pig is derived from a herd living in the wild in the Yucatan peninsula of Mexico (Panepinto L. M., Phillips R. W., Wheeler L. R., Will D. H. Lab. Anim. Sci. 1978; 28:308-313). Yucatan special mini-swine herd capable of expanding with minimal in-breeding to meet the large demand for organs required will be used, which will not allow the organs to continue to grow after the organ is transplanted into a human recipient. In preferred forms, the pigs to be genetically modified are Yucatan miniature pigs.

The blood groups of pigs are important issue to consider for xenotransplantation because some are also important transplantation antigens. Pig expresses antigens that correlate with human A or O blood group antigens. In some forms, pigs with A or O blood group antigens are used. In preferred forms, the donor pig is the same sex and compatible blood group as the organ recipient.

### B. Genetic Modifications

The genetic modifications can be made using any suitable technique, such as somatic cell nuclear transfer (SCNT) or CRISPR-Cas. In preferred forms, the disclosed limited genetic modifications in the Yucatan mini pigs do not weaken the pig genome fitness but will prevent hyperacute and delayed vascular rejection. In preferred forms, the donor pigs are genetically modified using somatic cell nuclear transfer (SCNT) and nucleases. For example, the pig genome can be modified using site-directed zinc finger nucleases (ZFNs) or TAL effector nucleases (TALENs) in somatic porcine cells, and cells carrying the desired genome modifications are then used for somatic-cell nuclear transfer (SCNT) to generate genetically modified piglets. In further preferred forms, genetically modifications are made by other methods in the future but the goal remains the same which is to have GM pig organs that function long term in the recipient.

In some forms, the genetically modified pigs have gene knockout in GGTA1, CMAH, and B4GALNT2. In preferred forms, the genetically modified pigs lack expression of genes GGTA1, CMAH, and B4GALNT2 and have transgene expression of human thrombomodulin, human CD46, and human CD55, and negative for PERV-C.

### 1. Exemplary Methods of Generating Genetically Modified Pigs

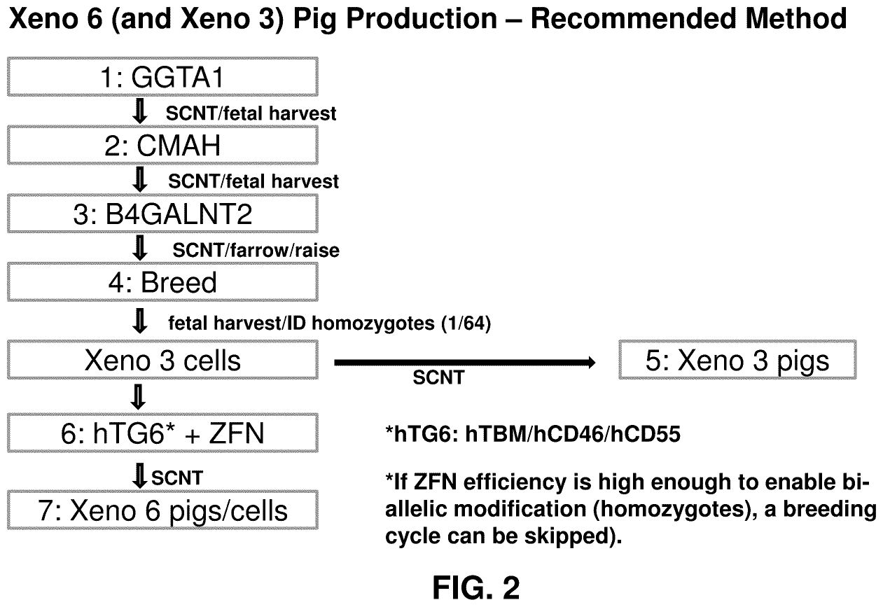

An exemplary method of generating genetically modified pigs is described below and the steps are outlined in FIG. 2. In preferred embodiments, the methods involve one or more of the following tasks and steps.

### TASK 1: Generating Male and Female GGTA+/− Fibroblasts Using Gene Targeting and Somatic Cell Nuclear Transfer (SCNT)

### Gene Targeting Reagent Development

Recombinant adeno-associated virus (rAAV) will be used to deliver a gene targeting construct to male and female Yucatan fetal fibroblasts. The GGTA targeting construct will contain an antibiotic resistance cassette (Neo^{R}, Blast^{R}, or similar) to facilitate identification of targeted cells.

### Infection and Selection

Male and female fetal fibroblasts will be infected with rAAV carrying the GGTA targeting construct. Standard selection and screening processes will be used to identify properly targeted cells. PCR, Southern blot, and DNA sequencing will be used to confirm proper targeting and rule out any random integration.

### Selection Marker Removal

The antibiotic selection marker will be flanked by loxP sites that will permit easy removal with Cre-recombinase. Properly targeted cells will be infected with rAAV-Cre and PCR, Southern blot, and DNA sequencing will be used to identify properly excised cells.

### SCNT for Fetal Harvest

Properly targeted cells will be used as nuclear donors for SCNT. Following transfer of reconstructed, nuclear transfer embryos, pregnancy establishment and maintenance will be monitored with ultrasound beginning at day 28 of gestation. At gestational day 35-40, fetal cell lines will be generated. Cell lines will be assessed using PCR, Southern blot, and DNA sequencing to confirm targeted genotype.

### TASK 2: Generating Male and Female GGTA+/−; CMAH+/− Fibroblasts Using Gene Targeting and SCNT.

### Gene Targeting Reagent Development

Recombinant adeno-associated virus (rAAV) will be used to deliver a gene targeting construct to male and female GGTA+/− Yucatan fetal fibroblasts. The CMAH targeting construct will contain an antibiotic resistance cassette (Neo^{R}, Blast^{R}, or similar) to facilitate identification of targeted cells.

### Infection and Selection

Male and female GGTA+/− fetal fibroblasts will be infected with rAAV carrying the CMAH targeting construct. Standard selection and screening processes will be used to identify properly targeted cells. PCR, Southern blot, and DNA sequencing will be used to confirm proper targeting and rule out any random integration.

### Selection Marker Removal

The antibiotic selection marker will be flanked by loxP sites that will permit easy removal with Cre-recombinase. Properly targeted cells will be infected with rAAV-Cre and PCR, Southern blot, and DNA sequencing will be used to identify properly excised cells.

### SCNT for Initial Pigs.

Properly targeted cells will be used as nuclear donors for SCNT. Following transfer of reconstructed, nuclear transfer embryos, pregnancy establishment and maintenance will be monitored with ultrasound beginning at day 28 of gestation. At gestational day 35-40, fetal cell lines will be generated. Cell lines will be assessed using PCR, Southern blot, and DNA sequencing to confirm targeted genotype.

### TASK 3: Generating Male and Female GGTA+/−; CMAH+/−; B4GALNT2+/− Fibroblasts and Pigs Using Gene Targeting and SCNT.

### Gene Targeting Reagent Development

rAAV will be sued to deliver a gene targeting construct to male and female GGTA+/−; CMAH+/− Yucatan fetal fibroblasts. The B4GALNT2 targeting construct will contain an antibiotic resistance cassette (Neo^{R}, Blast^{R}, or similar) to facilitate identification of targeted cells.

### Infection and Selection

Male and female GGTA+/−; CMAH+/− fetal fibroblasts will be infected with rAAV carrying the B4GALNT2 targeting construct. Standard selection and screening processes will be used to identify properly targeted cells. PCR, Southern blot, and DNA sequencing will be used to confirm proper targeting and rule out any random integration.

### Selection Marker Removal

The antibiotic selection marker will be flanked by loxP sites that will permit easy removal with Cre recombinase. Properly targeted cells will be infected with rAAV-Cre and PCR, Southern blot, and DNA sequencing will be used to identify properly excised cells.

### SCNT

Properly targeted cells will be used as nuclear donors for SCNT. Following transfer of reconstructed, nuclear transfer embryos, pregnancy establishment and maintenance will be monitored with ultrasound beginning at day 28 of gestation.

After full gestation (−114 days), initial litters will be farrowed. All pigs will be assessed using PCR, Southern blot, and DNA sequencing to confirm proper genotype.

### TASK 4: GGTA+/−; CMAH+/−; B4GALNT2+/− Males and Females Will be Raised to Breeding Age, Bred, and GGTA−/−; CMAH−/−; B4GAL−/− Fetal Fibroblasts Will be Obtained.

### Fetal Harvest and Identification of GGTA−/−; CMAH−/−; B4GALNT2−/− Male and Female Cells

Pregnancy establishment and maintenance will be monitored with ultrasound beginning at day 28 of gestation. At gestational day 35-40, fetal cell lines will be generated. Cell lines will be assessed using PCR, Southern blot, and DNA sequencing to confirm targeted genotype. Approximately 1 in 64 fetuses should have the GGTA−/−; CMAH-1−; B4GALNT2−/− genotype.

### TASK 5 (Optional): GGTA−/−; CMAH−/−; B4GALNT2−/−(Xeno 3) Male and Female Pigs Will be Produced by SCNT

scnt to Produce Xeno 3 Pigs

Cells generated in TASK 4 will be used as nuclear donors for SCNT. Following transfer of reconstructed, nuclear transfer embryos, pregnancy establishment and maintenance will be monitored with ultrasound beginning at day 28 of gestation.

### Birth of Xeno 3 Male and Female Pigs

After full gestation (−114 days), initial litters will be farrowed. All pigs will be assessed using PCR, Southern blot, and DNA sequencing to confirm GGTA−/−; CMAH−/−; B4GALNT2−/−(Xeno 3) genotype.

TASK6: Generating Male and Female GGTA−/−; CMAH−/−; B4GALNT2−/−; hTBM/hCD46/hCD55 Fibroblasts Using Gene Targeting and Somatic Cell Nuclear Transfer.

This TASK includes the use of zinc-finger nuclease (ZFN) to enhance gene targeting.

### Gene Targeting Reagent Development

ZFN-mediated gene targeting will be used to introduce the transgenes into the H11 or ROSA26 loci of a male and female GGTA−/−; CMAH−/−; B4GALNT2−/−. Note: H11 and ROSA26 are loci in the porcine genome associated with ubiquitous expression. The choice of locus will be based on ZFN reagent design. This strategy could allow the targeted insertion of transgenes into both alleles in a single step. This will involve the co-delivery of plasmid DNA carrying the hTBM/hCD46/hCD55 transgenes and the H11 or ROSA26 ZFNs. Targeting constructs will also contain Blast^{R} and Neo^{R} to facilitate identification of targeted cells.

### Transfection and Selection

Male and female GGTA−/−; CMAH−/−; B4GALNT2−/− fetal fibroblasts will be transfected with plasmid DNA carrying the hTBM/hCD46/hCD55 transgenes and the H11 or ROSA26 ZFNs. Standard selection and screening processes will be used to identify properly targeted cells. PCR, Southern blot, and DNA sequencing will be used to confirm proper targeting and rule out any random integration.

### Selection Marker Removal

The antibiotic selection marker will be flanked by loxP sites that will permit easy removal with Cre recombinase. Properly targeted cells will be infected with rAAV-Cre and PCR, Southern blot, and DNA sequencing will be used to identify properly excised cells.

### SCNT

Properly targeted cells will be used as nuclear donors for SCNT. Following transfer of reconstructed, nuclear transfer embryos, pregnancy establishment and maintenance will be monitored with ultrasound beginning at day 28 of gestation.

### Fetal Harvest and Possible First Xeno 6 Pigs

At gestational day 35-40, fetal cell lines will be generated. Cell lines will be assessed using PCR, Southern blot, and DNA sequencing to confirm targeted genotype. Any additional pregnancies may go to full term (˜114 days), producing possible Xeno 6 pigs for early experimentation. All pigs will be assessed using PCR, Southern blot, and DNA sequencing to confirm proper genotype.

TASK 7: GGTA−/−; CMAH−/−; B4GALNT2−/−; hTBM/hCD46/hCD55 Pigs (Xeno 6) Pigs Will be Produced by SCNT.

### SCNT to Produce Xeno 6 Pigs

Cells generated in TASK 6 will be used as nuclear donors for SCNT. Following transfer of reconstructed, nuclear transfer embryos, pregnancy establishment and maintenance will be monitored with ultrasound beginning at day 28 of gestation.

### Birth of Xeno 6 Male and Female Pigs

After full gestation (˜114 days), initial litters will be farrowed. All pigs will be assessed using PCR, Southern blot, and DNA sequencing to confirm GGTA−/−; CMAH−/−; B4GALNT2−/−; hTBM/hCD46/hCD55 pigs (Xeno 6) genotype.

### IV. Methods of Inducing Tolerance

Methods for inducing tolerance in a transplant organ recipient to an organ graft from the donor animals are described. The first step in inducing tolerance is when the GM organ graft is populated by recipient's lymphocytes, stem cells, and factors, which are introduced within a donor animal when it is in a form of a developing pig fetus. During this time the lymphocytes and stem cells differentiate and mature into regulatory cells, suppressor cells, select B cells, and antibodies which after delivery of the pig fetus, are enriched and expanded ex-vivo. Another potential benefit of infusing human cells and factors into the fetal pig, is the potential for accommodation of the pig endothelial cells to human proteins which may reduce the potential metabolic incompatibilities associated with protein/ligand differences between species. These cells and factors are then given back to the human recipient who donated the original lymphocytes and stem cells prior to receiving the pig organ allowing the xenotransplant to occur without the need for chronic immunosuppression. Generally, the method includes the steps of obtaining a plurality of cells and factors from the transplant organ graft recipient, and allowing those cells to differentiate and mature in a fetal donor animal to generate cells and factors that are tolerant to the tissues and cells of the fetal donor animal. These cells originally derived from the organ recipient are then collected from the developed donor animal, and infused back into the same transplant organ recipient. After the transfer of these cells back into the transplant organ recipient, an organ graft is then collected from the developed same donor animal and transplanted into the transplant organ graft recipient.

More particularly, the present invention provides a cell population with immune regulatory functions, including immune tolerant lymphocytes, stem cells, and soluble factors, for use in inducing tolerance in a transplant organ recipient. When the recipient's lymphocytes and stem cells are transferred from the immune competent donor animal back to the recipient, they reduce the likelihood of rejection of the graft organ by the transplant organ recipient.

Reconstitution of the graft with cells from the organ recipient, including dendritic cells, macrophages, lymphocytes, stem cells, plasma cells and endothelial cells are described with the modification occurring outside of the intended organ graft recipient. The method provides organ grafts that are less susceptible to rejection by repopulating the organ with cells from the intended transplant organ recipient. Acceptance of the donor organ graft by the recipient is enhanced by replacement and repopulation of the organ graft by cells from the intended organ graft recipient.

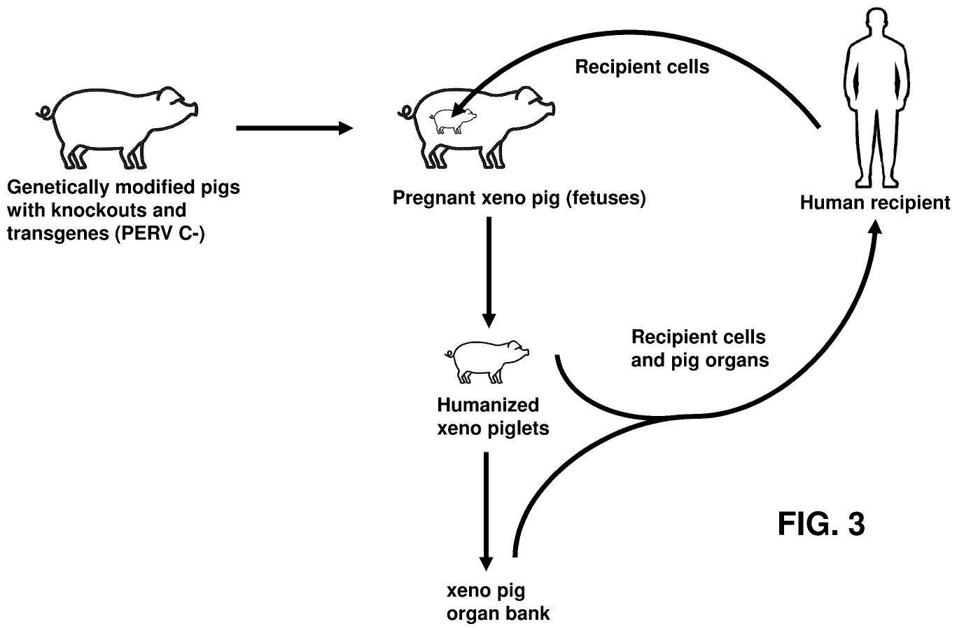

The use of cell therapy between human recipients and pig donors (transferring lymphocytes, stem cells and factors from the organ recipient and having those cells populate the GM donor fetus organs and then after delivery of the GM donor piglet and expansion and purification of these recipient's lymphocytes and stem cells, these cells are transferred back to the organ recipient prior to organ transplantation) allows a more humanized pig organ to be transplanted, as the pig organ has been repopulated with the recipient cells, such that the organ recipient develops tolerance and avoids the need for chronic immunosuppression. Any exemplary flow chart of the steps involved in the cell therapy for inducing tolerance is shown in FIG. 3.

In exemplary forms, the methods of inducing tolerance using genetically modified (GM) pigs and organs derived therefrom for xenotransplantation that are personalized to a specific organ recipient and that do not require chronic immunosuppression in the recipient following xenotransplantation.

Typically, the methods include one or more steps including:
(i) transferring cells and factors of the organ recipient (e.g., human) into a genetically modified (GM) donor pig fetus in utero,
(ii) administering, after completion of gestation of the GM donor pig fetus and delivery and some development of the resultant GM donor pig, the recipient's cells and factors of the GM donor pig back into the organ recipient; and
(iii) transplanting, 5 to 9 days following the transferring of cells and factors of the GM donor pig into the organ recipient, an organ from the same GM donor pig into the organ recipient; and
(iv) optionally storing the organ from the GM donor pig prior to transplanting into the recipient or storing at pig organ bank.

A preferred animal is a pig. A preferred species of pig is a Yucatan pig.

Each of these steps is described in more detail, below.

A. Transfer Cells and Factors of the Organ Recipient into the Donor Animal in Utero

In some forms, the first step for inducing tolerance includes transplantation of cells (e.g., lymphocytes, stem cells, and APC) and factors from the organ graft recipient into the donor animal. In preferred forms, the cells and factors of the organ recipient are transferred to the genetically modified donor pig while the donor pig is a donor pig fetus in utero in the parent pig.

The methods typically transfer cells including lymphocytes, stem cells, and other factors from a subject that is the intended organ recipient. Therefore, in some forms, the methods include one or more steps of extracting cells including viable lymphocytes, stem cells, and other factors from a subject, and optionally storing the viable cells and other factors in a manner suitable for maintaining viability prior to transferring into the donor pig. Techniques for extracting and storing viable cells and other factors from a subject are known in the art.

Typically, the donor pig is a genetically modified fetus, such as a fetus in utero within a mother pig. In some forms, the mother pig is also a genetically modified pig. For example, in some forms, the donor pig fetus is being gestated in a genetically modified parent pig, where the genetic modifications of the donor pig fetus are the same as the genetic modifications of the parent pig. Techniques for transferring viable cells and other factors from a subject into a pig in utero are known in the art.

In some forms, lymphocytes, hematopoietic and mesenchymal stem cells, bone marrow, or blood and other factors are infused into the abdominal cavities or thymus of a fetal animal. In some forms, the infusion is performed near the end of the first trimester or during the beginning of the second trimester of gestation of the donor animal.

In some forms, cells to be infused into the donor animal include lymphocytes, stem cells and other factors which will be obtained from the organ recipient's peripheral blood and bone marrow.

Generally, prior to transferring, the cells and factors of the subject are collected from blood and bone marrow of the organ recipient and (ii) are partially depleted of CD4+ and CD8+ T cells. Reduction in the number of T cells from the organ recipient in the infusion may be necessary to avoid any development of graft-vs-host disease. Thus, in further forms, prior to transferring, the cells, and factors of the organ recipient (i) are collected from blood and bone marrow of the organ recipient and/or (ii) are partially depleted of CD4+ and CD8+ T cells. In some embodiments, 30%, 40%, 50%, 60%, 70%, 80%, 90%, 95%, or more than 95% of the CD4+ and CD8+ T cells are depleted prior to being transferred into the donor pig fetus in utero. In preferred embodiments, about 40%-60%, for example, about 50%, of the CD4+ and CD8+ T cells are depleted prior to being transferred into the donor pig fetus in utero.

In exemplary forms, the transferring is accomplished by injecting the partially depleted cells and factors of the organ recipient into donor pig fetus, for example, by injection into the abdominal cavity of a genetically modified donor pig fetus in utero. Typically, the genetic modifications are limited to three gene knockouts and, optionally, three human transgenes. Typically, the GM donor pig fetus is the same sex and compatible blood group as the organ recipient and preferably has low anti-pig antibodies.

The proliferation, maturation, and differentiation of the organ recipient's cells within the donor animal (or donor fetus) may optimally be enhanced by incubating the organ graft recipient's cells with growth factors prior to infusion into the donor animal and/or using growth factors in the pig when it is in utero or after delivery. For example, the cells could be incubated with recombinant human GM-CSF, IL1, IL3, IL6, IL7, growth hormone, or insulin-like growth factors, etc. or a combination of factors. In some forms, the factors or a combination of factors can be given to the GM pigs in utero or after delivery.

Thus, in preferred forms, antigen specific regulatory cells and factors responsible for tolerance of the organ graft recipient's immune system are introduced to the donor antigens (or organ donor antigens) by intrauterine induction of tolerance in the fetal organ donor. In further preferred forms, the antigen specific regulatory cells and factors are capable of blocking or inhibiting the reaction of differentiated lymphocytes and factors of the organ graft recipient to the cells of the organ donor. Therefore, the induction of antigen specific tolerance enhances the development of suppressor or regulatory T cells and associated factors, veto cells, enhancement factors, and anti-idiotype antibodies.

Accordingly, to achieve the induction of antigen specific tolerance and long-term survival of the organ graft, recipient lymphocytes and antigen presenting cells in the donor animal will be attained without the development of significant GvHD.

### B. Monitoring Development of the Donor Animals

Following infusion of the cells from the organ recipient, the donor pigs are monitored to establish chimerism, i.e., differentiation and maturation of the lymphocyte populations, stem cells, and factors from the organ graft recipient within the donor pigs, as well as tolerance of the lymphocyte populations and factors to the organ donor antigens. The assays used to select the best or most optimal donor pig for providing tolerant lymphocytes, stem cells and best suited factors will be readily apparent to the skilled person.

The monitoring involves a quantitation of the extent of chimerism in the donor animal, i.e., the relative numbers of organ recipient cells within the donor animal, and monitoring of the tolerance for the organ recipient cells towards the donor antigens.

Exemplary assays for monitoring include, but not limited to:
(1) flow cytometry using antibodies specific for the donor and/or for the organ recipient species, e.g., monoclonal antibodies to human CD45, CD2, CD3, CD5, CD7, CD19, HLA-DR, HLA-ABC, CD45RO, CD45RA, CD4, CD8, CD34, and CD31 as well as to swine CD45, CD2, CD3, CD4, CD8 and swine surface immunoglobulin;
(2) immunohistochemistry to establish chimerism with biopsies of tissues or organs from the donor animals such as lymph node, spleen, and thymus;
(3) in vitro tests of tolerance such as in vitro maturation assays of T cells and/or B cells from the recipient against donor cells; and
(4) in vivo tests of tolerance in the donor animals, i.e., an “immune challenge.”

Chimerism may be readily followed using flow cytometry and antibodies specific for the donor and for the organ recipient species. For example, if the animal is a pig, then pig blood and bone marrow may be screened for the relative numbers of human lymphocytes as well as pig lymphocytes. The antibodies may include monoclonal antibodies to human CD45, CD2, CD3, CD5, CD7, CD19, HLA-DR, HLA-ABC, CD45RO, CD45RA, CD4, CD8, CD34, and CD31 as well as to swine CD45, CD2, CD3, CD4, CD8 and swine surface immunoglobulin. The degree of chimerism may also be quantified in lymphoid tissues, including the thymus, the spleen, and the lymph nodes. In a preferred embodiment, the method of the present invention further includes characterizing the extent of chimerism within the tissue to be used as an organ graft. These studies would include immunohistochemistry, such as immune alkaline phosphatase stains of biopsy tissues using antibodies to human factor VIII, dendritic cells, CD45, CD4, CD8, CD2, CD5, HLA-DR, and HLA-ABC.

Immune tolerance of the cells originated from the organ recipient, differentiated and matured in the donor animal, towards the donor organs and tissues may be monitored by a variety of methods. One method of monitoring immune tolerance involves taking biopsies of the donors' tissues intended to be grafted, and characterizing the donors' tissues for evidence of a rejection process by the recipient cells. Other tissues of the donors, such as skin, liver, and intestines, may also be examined for evidence of GvHD. Another method of monitoring involves performing in vitro tests of tolerance, where the in vitro tests may include mixed lymphocyte cultures (MLC) and suppressor cell assays. In such in vitro tests, cultured lymphocytes are added to an MLC of fresh organ recipient responder cells versus organ donor stimulator cells. Because some xenograft pairs may show a limited proliferation, limiting dilution assays may be necessary to establish a reduction in precursor cytotoxic T lymphocytes. An additional method of monitoring involves using flow cytometry and immunohistochemistry studies to provide a relative quantitation of lymphocytes with a phenotype for suppressor cells; for example, antibodies to CD31 may be used. The organ graft recipient cells may also be monitored for a reduction of T cell receptor rearrangements corresponding to lymphocytes reactive against organ donor antigens.

The use of a pig donor for the development of tolerance provides the opportunity for a novel bioassay for assessing immune tolerance. An advantage of using donors as bioassays includes assessing the degree of immune tolerance in the donors prior to transplanting the organ graft from a donor to a recipient. Chimeric like donors are infused with fresh lymphocytes from the organ graft recipient, and, if the chimeric like animal is truly tolerant, then regulatory cells and factors prevent the fresh lymphocytes from causing either GvHD or immune rejection of the intended graft tissue. The above bioassay using the donors is referred to as an “immune challenge”. Following the challenge, biopsies may be taken of tissues routinely injured by GvHD, and biopsies of the intended graft tissue can also be taken. Besides monitoring the development of tolerance, the challenge provides a further benefit to the degree that it stimulates the expansion of regulatory cells; for example, the development of additional suppressor T cells may be stimulated.

In preferred embodiments, assays for chimerism and determinations of chimerism in cell suspensions are performed on the pig donors. At 0 to four months after birth of the chimeric like animal, blood and bone marrow specimens are collected from the donor animals. Preferably, the buffy coats of the specimens are isolated, and the cells are stained with antibodies specific for CD45 of the species of the organ graft recipient. The extent of chimerism may be quantified using analytical flow cytometry. Evidence for maturation of T cells may be established using double label flow cytometry for CD4 and CD8, for single positive cells, and expression of CD3. T cells with a phenotype for suppressor cells may be quantified with antibodies to CD31 and optionally Leu15. Maturation of B lymphocytes may be assayed with CD19 and CD21, and macrophages may be assayed with antibodies to CD14, and CD11b. Additional studies may be performed on the best or most optimal donor animals by making cell suspensions of lymph node biopsies, and by making fine needle aspirates of the spleen. Optionally, chimerism may be assessed in cell suspensions using cytogenetics and restricted length fragment polymorphisms (RLFP). Immunopathology of the donor tissues may be conducted to establish chimerism and to rule out GvHD and immune reactions to the graft tissue. Immunopathology studies may be performed on biopsies of the skin, the liver, the intestines, the bronchial mucosa, the thymus, the lymph nodes, the spleen, and/or other tissues from the intended graft. These target tissues are stained and evaluated for cellular injury indicative of GvHD. Using antibodies specific for subgroups of cells of the organ graft recipient species, immunohistochemistry can establish chimerism with biopsies of lymph node, spleen, and thymus. In general, mouse monoclonal antibodies against human CD45, CD4, CD8, CD3, CD19, CD21, CD31, perforin, HLA-DR, HLA-DQ, HLA-ABC, CD14, CD11b and CD1 are usually used to identify mature T cells, suppressor T cells, B cells, macrophages, and dendritic cells. CD1 also identifies thymic dendritic cells and cutaneous Langerhans cells. Antibodies to factor VIII of the organ graft recipient species typically identify reconstitution of the endothelium with organ graft recipient cells. Sections of tissue are typically incubated with a primary antibody; washed; incubated with a secondary antibody; for example, biotinylated horse anti-mouse immunoglobulin; and developed using the avidin-biotin complex assay. Immunofluorescence stains may be performed for organ graft recipient species immunoglobulins and for deposits of complement of either the organ graft recipient or the donor species.

C. Transfer Recipient's Cells and Factors Expanded and Matured in the Developed GM Donor Pig Back into the Organ Recipient

In some forms, after delivery of the donor pig fetus and some development of the resultant donor pig, cells and factors of the donor pig are collected and transferred back into the organ recipient.

Therefore, the methods require the passage of time between steps (a) and (b) sufficient for the development of the pig fetus in utero, and optionally for the delivery of the GM donor pig as a piglet, and further optionally for the growth and development of the piglet. Typically, the growth and development does not exceed one week, one month, two months, three months, four months, five months, six months, or one year following delivery of the donor pig. In preferred forms, the growth and development does not exceed six months following delivery of the donor pig.

In some forms, the donor pigs are monitored for several months after birth for chimerism and absence of GvHD. A best or most optimal donor pig is chosen from the number of donor pigs, and the organ graft recipient cells and factors are harvested from the best donor pig.

In some forms, the cells and factors are collected from blood, bone marrow, cord blood, spleen, and lymph nodes of the donor pig. In other forms, cells and factors of the donor pig are incubated in vitro with a combination of factors for expansion prior to infusion back to the organ recipient.

These cells and factors are subject to (i) enrichment for cells and factors originated from the organ recipient, i.e., cells that are derived from those transferred from the organ recipient into the donor pig fetus in utero, and (ii) removal or depletion of cells of donor pig.

Typically, prior to administering, cells and factors originated from the organ recipient are subject to further enrichment for select subpopulations of cells, including one or more of the following:
(i) enrichment for foxP3+CD4+CD3+CD25+ natural T regulatory cells,
(ii) enrichment for adaptive CD4+CD49b+LAG3+CD226+ regulatory Tr1 cells,
(iii) enrichment for CD4+CD25-CD69+foxP3-LAP+(TGFβ-producing type 3 helper T cells), and
(iv) enrichment for PD-L1 human dendritic cells.

In some forms, one or more of these subpopulations of cells are subsequently cultured, expanded, and purified.

Regulatory T cells (Tregs) are known to restrain immune responses to self-antigens, non-self-antigens, and associated inflammation (Cook L., Stahl M., Han X., et al., Gastroenterology. 2019; 157(6):1584-1598). Previous studies indicated that Tregs' adoptive transfer is an immunomodulatory therapy to prevent type 1 diabetes, autoimmune diseases, graft-versus-host disease (GVHD), and rejection after organ transplantation. Recently, accumulated evidence indicated that adoptive transfer with antigen-specific Tregs prevents xenograft rejection by downregulating the immune responses of effector T cells and tissue injuries by exerting on-targeted suppression function (Yi S., Ji M., Wu J., et al., Diabetes. 2012; 61(5):1180-1191; Putnam A. L., Safinia N., Medvec A., et al., American Journal of Transplantation. 2013; 13(11):3010-3020).

FoxP3⁺CD4⁺CD3⁺CD25⁺T (Treg) cells are a small subset of CD4 T cells with unique immune regulatory function that are indispensable in immunity and tolerance. In some forms, these cells and factors originated from the organ recipient are enriched for foxP3⁺CD4⁺CD3⁺CD25⁺ natural T regulatory cells, to about 2-fold, 3-fold, 4-fold, 5-fold, 6-fold, 7-fold, 8-fold, 9-fold, 10-fold, or more than 10-fold, preferably, between about 5-fold and 10-fold, compared to the same population of cells prior to the enrichment step. In some forms, these FoxP3+CD4+CD3+CD25+ natural T regulatory cells originated from the organ recipient are cultured and expanded, to about 2-fold, 3-fold, 4-fold, 5-fold, 6-fold, 7-fold, 8-fold, 9-fold, 10-fold, or more than 10-fold, preferably, between about 5-fold and 10-fold, compared to the same population of cells prior to the expansion step.

Type 1 regulatory T (Tr1) cells, a major class of Tregs, are characterized by the secretion of high levels of interleukin-10 (IL-10) and the co-expression of surface markers LAG-3 and CD49b without constitutive expression of FOXP3 and CD25, which have immune-suppressive potency and bear alloantigen specificity (Roncarolo M. G., et al., Immunological Reviews. 2006; 212(1):28-50). IL-10 can control the differentiation and proliferation of Tregs and maintain peripheral T cell tolerance. Extensive studies demonstrated that Tr1 cells, which represent the major subset of the regulatory T cell population, can reverse tissue damage and increase transplant survival in GVHD (Heinemann C., Heink S., Petermann F., et al., Nature Communications. 2014; 5(1): p3770; Zhang P., Lee J. S., Gartlan K. H., et al., Science immunology. 2017; 2(10)). Studies in transplantation animal models and clinical trials demonstrate that alloantigen-specific Tregs have superior antigen-specific efficacy compared with polyclonally exposed Tregs and can achieve targeted suppression and prevent allograft tissue damage, thereby reducing the risk in transplantation (Barrat F. J., Cua D. J., Boonstra A et al., The Journal of Experimental Medicine. 2002; 195(5):603-616).

Thus, in some forms, these cells and factors originated from the organ recipient are enriched for regulatory Tr1 cells, i.e., adaptive CD4⁺CD49b⁺ LAG3⁺CD226⁺ regulatory Tr1 cells. In some forms, these adaptive CD4⁺CD49b⁺ LAG3⁺CD226⁺ regulatory Tr1 cells originated from the organ recipient are cultured and expanded. In some forms, these cells and factors originated from the organ recipient after residing inside the donor animal are enriched for adaptive regulatory Tr1 cells, to about 2-fold, 3-fold, 4-fold, 5-fold, 6-fold, 7-fold, 8-fold, 9-fold, 10-fold, or more than 10-fold, preferably, about 5-10 fold, compared to the same population of cells prior to the enrichment step. In some forms, these adaptive CD4⁺CD49b⁺ LAG3⁺CD226⁺ regulatory Tr1 cells originated from the organ recipient are cultured and expanded, to about 2-fold, 3-fold, 4-fold, 5-fold, 6-fold, 7-fold, 8-fold, 9-fold, 10-fold, or more than 10-fold, preferably, between about 5-fold and 10-fold, compared to the same population of cells prior to the expansion step.

Type 3 helper T cells (Th3) are a CD4+CD25-CD69+foxP3-LAP+ TGF beta producing regulatory T cell subset that has been found to be important in intestinal immune regulation. Th3 cells are identified by the production of high amounts of TGFβ1 when stimulated with their specific antigen. In some embodiments, these cells and factors originated from the organ recipient after residing inside the donor animal are enriched for TGFβ-producing type 3 helper T cells, to about 2-fold, 3-fold, 4-fold, 5-fold, 6-fold, 7-fold, 8-fold, 9-fold, 10-fold, or more than 10-fold, preferably, between about 5-fold and 10-fold, compared to the same population of cells prior to the enrichment step. In some forms, these TGFβ-producing Th3 cells originated from the organ recipient are cultured and expanded, to about 2-fold, 3-fold, 4-fold, 5-fold, 6-fold, 7-fold, 8-fold, 9-fold, 10-fold, or more than 10-fold, preferably, between about 5-fold and 10-fold, compared to the same population of cells prior to the expansion step.

In further embodiments, these cells and factors originated from the organ recipient after residing inside the donor animal are enriched for organ recipient PD-L1 human dendritic cells. In other forms, these PD-L1 human dendritic cells originated from the organ recipient are cultured and expanded as the cells described above and purified.

In some forms, the organ recipient subject is administered an immunomodulatory agent to suppress the immune system in the subject. Preferred immunomodulatory agents include Cyclophosphamide +/− Fludarabine. Typically, the methods to administer the immunomodulatory agent or agents to the subject in an effective amount and at a time to achieve immunomodulatory effects in the subject, prior to the administering the cells and factors that were originated from the organ recipient and have had conditioning in the donor animal. For example, in some forms, the methods administer Cyclophosphamide and/or Fludarabine to the subject in an amount sufficient to achieve immunomodulatory effects 5 to 9 days prior to the step of administering the cells and factors. Plasmapheresis may also be considered where it involves removing blood through a needle or catheter and circulating it through a machine where the blood is separated into the blood elements and plasma. Plasmapheresis is often performed before a transplant to remove antibodies and abnormal blood proteins against the donor from the recipient. Thus, in some forms, plasmapheresis is carried out in the receipt prior to the step of administering the cells and factors.

### D. Organ Transplantation

Once presumed tolerance in the organ graft recipient is confirmed, the graft from the GM donor pig is transplanted into the organ graft recipient. Typically, the step of transplanting is caried out from about 5 to about 9 days after the transferring of the recipient's cells and factors from the GM donor pig back into the organ recipient.

Methods for organ transplantation and preservation are known in the art. Surgical transplantation techniques are well known in the art (see, e.g., Simmons, et al., “Transplantation,” in Schwartz, et al., 1989, eds. Principles of Surgery, McGraw-Hill, N.Y., pp. 387-458). The organ graft recipient is monitored for evidence of rejection of the organ graft in accordance with routine practice in the art (tissue biopsy, donor derived cell free DNA, molecular microscopic diagnostic system, next generation sequencing and SLA/HLA antibodies), but the need for immunosuppressive therapy is significantly reduced, preferably avoided due to the development of tolerance, compared to known methods of transplantation in the art.

If the recipient needs a second organ transplant after a xenotransplant, then the other organs from the same donor pig would be available in the future pig organ bank for subsequent transplantation.

Transplantation in accordance with methods of inducing immune tolerance as described herein will significantly reduce rejection for a variety of solid tissue organs and tissues including skin, heart, kidneys, liver, lungs, intestines, pancreas, pancreatic islets, retina, cornea, and bone. For example, a common cause of visual impairment in the aged is macular degeneration with degeneration of the retinal pigment epithelial cells. After inducing tolerance of the patient to the donor animal, the retina of the donor animal can be transplanted into the patient with reduced risk of rejection. In preferred embodiments, transplantation in accordance with the disclosed methods will not require long-term use of immunosuppressive drugs in the organ **recipients (preferably human recipients), thus reducing rate of infection due to chronic immunosuppression as well as the cost and complications associated with use of these drugs.

In some embodiments, anti-inflammatory drugs are used in the organ recipients post-xenotransplant for 6-12 months.

### E. Organ Bank

The donor organs can be harvested and preserved by standard techniques using either cold static storage, machine perfusion or a combination of both depending on the organ involved. If the organ will be placed in a storage bank, organ preservation will be somewhat different. For example, in the case of pig organ donors, the pig will be placed on cardiopulmonary bypass and cooled slowly. The perfusate will be changed to a preservation solution containing basic nutrients, vitamins, amino acids, glutamine, electrolytes, colloid, buffers, antioxidants, antimicrobial agents, free radical scavengers, anti-inflammatory agents, insulin, glucose, mannitol, PGE1, heparin, oxygen, and cryoprotectants. When the core temperature of the pig reaches 4 degrees C., cardiopulmonary bypass will be stopped, and the pig organs will be removed and placed in a refrigerator where they will be slowly cooled to subzero temperatures and maintained indefinitely. When the pig organ is needed for xenotransplantation, it will be transported to a transplant facility where it will be slowly re-warmed and when it reaches a certain temperature, standard preservation solutions for that particular organ will be injected and the organ then placed on machine perfusion to make it ready for xenotransplantation (e.g., pancreatic islets will be harvested from the pancreas by current state of the art).

### V. Methods of Use

Methods of using the viable cells and organs derived from a donor pig according to the above-described method are also provided.

The cells and organs can be used for any methods that require viable cells and organs. In some forms, the cells and organs are used for methods of treatment. Therefore, methods of treating a subject having a disease or disorder, including transplanting into the subject an organ or cell produced according to the described methods are provided. Typically, the methods are effective to treat or prevent the disease or disorder in the subject. In some forms, the disease or disorder is selected from heart diseases, kidney diseases, liver diseases, lung diseases, eye diseases, and pancreatic diseases. In other forms, the disease or disorder is a congenital disorder or condition of one or more organs such as congenital heart disease, congenital kidney disease, congenital liver disease, congenital eye disease, etc.

### A. Recipient Subjects

Recipient subjects are described. Typically, the subject is a human, such a human patient in need of treatment for a disease or disorder and organ failure. In some forms, the recipient has been identified as having, or as being at risk of having a disease. The disease can be chronic or acute. Exemplary diseases include congenital diseases of one or more organs or tissues. For example, in some forms, the recipient is a subject having a disease that causes end-stage failure of an organ, necessitating transplantation. In some forms, the recipient subject has a disease or disorder that is known in the art to be successfully treated by an allograft transplant.

In some forms, the recipient is a subject having diabetes mellitus, such as type 1 diabetes or type 2 diabetes. In some forms, the recipient subject is dependent upon external insulin or insulin pump.

In some forms, the recipient is a subject having a liver disease or disorder, such as hepatitis. Exemplary liver diseases include hepatitis C, diseases caused by drugs, poisons, or alcohol, such as fatty liver disease and cirrhosis, liver cancer and congenital diseases, such as hemochromatosis and Wilson disease. In some forms, the recipient subject is dependent upon antiviral medication.

In some forms, the recipient is a subject having a kidney disease or disorder, such as renal failure. In some forms, the recipient subject is dependent upon dialysis. Exemplary kidney diseases include chronic kidney disease, kidney stones, glomerulonephritis, polycystic kidney disease, atypical hemolytic uremic syndrome (aHUS), alport syndrome, amyloidosis, cystinosis, Fabry disease, focal segmental glomerulosclerosis (FSGS), and goodpasture syndrome.

In some forms, the recipient is a subject having a heart disease or disorder. Exemplary heart diseases include coronary heart disease, cardiomyopathy, or congenital heart disease. In some forms, the recipient is a subject identified as having had or at increased risk of myocardial infarction. In some forms, the recipient subject is dependent upon a pacemaker and/or blood thinners and mechanical heart pump.

In some forms, the recipient is a subject having a neurodegenerative disease or disorder. The term neurodegenerative disease is an umbrella term for a range of conditions which primarily affect the neurons in the human brain. Many central nervous system disorders including stroke, traumatic brain injury, and spinal cord injury can also be linked to neurodegenerative diseases through common underlying mechanisms of nerve cell damage and destruction. Exemplary neurodegenerative diseases include, but are not limited to, Parkinson's disease (PD) and PD-related disorders.

In some forms, the recipient is one who has previously undergone one or more transplants. For example, in some forms, the recipient subject is one who has previously had an autoimmune response, or an inflammatory response associated with a previous transplant. In some forms, the recipient has previously undergone transplant rejection. In some forms, the recipient has been identified as having graft vs. host disease (GvHD).

In some forms, the recipient is an adult. In other forms, the recipient is a child, such as an infant. In some forms, the recipient is an infant identified as having been born with a disease or disorder that requires a transplant.

The disclosed compositions and methods can be further understood through the following numbered paragraphs.

1. A method of producing and using genetically modified (GM) pig organs for xenotransplantation that are personalized to a specific organ recipient and that do not require chronic immunosuppression following xenotransplantation, the method comprising:
(a) transferring cells and factors of the organ recipient into a GM donor pig fetus in utero,
   wherein, prior to transferring, the cells and factors of the organ recipient (i) are collected from blood and bone marrow of the organ recipient and (ii) are partially depleted of CD4+ and CD8+ T cells,
   wherein the transferring is accomplished by injecting the partially depleted cells and factors of the organ recipient into the abdominal cavity of the GM donor pig fetus, wherein the donor pig fetus is a genetically modified donor pig fetus being gestated in a genetically modified parent pig, wherein the genetic modifications of the donor pig fetus are the same as the genetic modifications of the parent pig, wherein the genetic modifications are limited to three gene knockouts and, optionally, three human transgenes, and wherein the GM donor pig fetus is the same sex and compatible blood group as the organ recipient;
(b) transferring, after completion of gestation of the GM donor pig fetus and delivery and some development of the resultant GM donor pig, the organ recipient's cells and factors from the GM donor pig back into the organ recipient,
   wherein, prior to transferring, the cells and factors of the GM donor pig (i) are collected from one or more of blood, bone marrow, spleen, cord blood and lymph nodes of the GM donor pig, (ii) are depleted of donor pig cells, and (iii) are subject to enrichment, expansion, and/or purification for the organ recipient's cells selected from the group consisting of foxP3+CD4+CD3+CD25+ natural T regulatory cells, adaptive CD4+CD49b+LAG3+CD226+ regulatory Tr1 cells, CD4+CD25-CD69+foxP3-LAP+(TGFβ-producing type 3 helper T cells), and organ recipient PD-L1 human dendritic cells,
   wherein, 5 to 9 days prior to transferring, the organ recipient is administered Cyclophosphamide and/or Fludarabine, and
(c) transplanting, 5 to 9 days following the transferring of the organ recipient's cells and factors from the GM donor pig back into the organ recipient, an organ from the same GM donor pig into the organ recipient.

2. The method of paragraph 1, further comprising administering one or more anti-inflammatory drugs to the organ recipient post-xenotransplant.

3. The method of paragraph 2, wherein the one or more anti-inflammatory drugs are administered to the organ recipient for 6-12 months.

4. The method of any one of paragraphs 1 to 3, wherein the organ recipient is a subject having been diagnosed as having a disease or disorder selected from the group consisting of heart diseases, kidney diseases, liver diseases, lung diseases, eye diseases, pancreatic diseases, and congenital diseases of one or more organs.

5. The method of any one of paragraphs 1 to 4, wherein the organ from the GM donor pig comprises all or part of a heart, bone, liver, lung, kidney, pancreas, intestine, or eye.

6. The method of any one of paragraphs 1 to 5, wherein the organ from the GM donor pig comprises one or more cell types selected from the group consisting of skin and bone cells (e.g., fibroblasts, osteoblasts, osteocytes, osteoclasts, osteoprogenitor cells), cardiac cells (e.g., fibroblasts (CFs), cardiomyocytes, smooth muscle cells (SMCs), and endothelial cells (ECs)), blood cells (e.g., red blood cells, white blood cells), liver cells e.g., (hepatocytes (HCs), hepatic stellate cells (HSCs), Kupffer cells (KCs), and liver sinusoidal endothelial cells (LSECs)), kidney cells (e.g., glomerular basement membrane/glomerular endothelial cells, macula densa cells, mesangial cells, podocytes, tubule epithelial cells), cells of the pancreas (e.g., islet cells, including alpha, beta and delta cells), and stem cells.

7. The method of any one of paragraphs 1 to 6, wherein the GM donor pig is at the same site as the recipient during the transplanting in step (c).

8. The method of any one of paragraphs 1 to 7, wherein the GM donor pig and the recipient are not at the same site during the transplanting in step (c), and
wherein the method further comprises one or more steps for storing and transporting the organ from the donor site to the recipient site, optionally via a pig organ storage bank.

9. The method of any one of paragraphs 1 to 8, wherein the donor pig is a species selected from Sus scrofa domesticas, Sus ahoenobarbus, Sus barbatus, Sus bucculentus, Sus cebifrons, Sus celebensis, Sus heureni, Sus oliveri, Sus philippensis, and Sus verrucosus.

10. The method of any one of paragraphs 1 to 9, wherein the pig is a Sus scrofa domesticas pig.

11. The method of paragraph 10, wherein the pig is a Yucatan pig.

12. The method of any one of paragraphs 1 to 11, wherein the cells of the organ recipient in step (a) are selected from the group consisting of lymphocytes and stem cells.

13. The method of any one of paragraphs 1 to 12, wherein the pig organ after transplanting in the recipient grows less than 5%, 10%, 15%, or 15% in volume.

14. The method of any one of paragraphs 1 to 13, wherein the donor pig is chosen so that the pig organ will not continue to grow in the recipient after transplantation.

15. The method of any one of paragraphs 1 to 14, wherein the GM pig comes from a GM pig population was expanded with minimal in-breeding.

16. The method of any one of paragraphs 1 to 15, wherein the genetic modifications to the donor pigs are limited to fewer that seven genetic modifications.

17. The method of any one of paragraphs 1 to 16, wherein the genetic modifications are made using Somatic Cell Nuclear Transfer (SCNT), CRISPR-Cas, or other methods.

18. The method of any one of paragraphs 1 to 17, wherein the genetic modifications in the pig include knockouts of 3 pig genes and the addition of 3 human transgenes.

19. The method of any one of paragraphs 1 to 18, wherein Porcine Endogenous Retrovirus-C (PERV-C) is eliminated in the pig organ.

20. The method of any one of paragraphs 1 to 19, wherein the GM pigs are raised in a pathogen-free environment, are tested for infectious agents of concern, and are cleared of all viruses of concern.

21. The method of any one of paragraphs 1 to 20, wherein the recipient has a more personally matched GM pig, wherein the pig organ in the GM pig is repopulated with the recipient's cells to make the pig organ more humanized.

22. The method of any one of paragraphs 1 to 21, wherein the removal of the recipient's lymphocytes comprises the removal of hematopoietic and mesenchymal stem cells and other factors from the recipient's blood and bone marrow.

23. The method of any one of paragraphs 1 to 22, wherein the injection of the recipient's lymphocytes comprises the injection of the recipient's hematopoietic and mesenchymal stem cells and other factors into the donor pig in utero.

24. The method of any one of paragraphs 1 to 23, wherein the recipient's lymphocytes and hematopoietic and mesenchymal stem cells injected into the donor pig in utero are tracked.

25. The method of any one of paragraphs 1 to 24, wherein the recipient's lymphocytes and stems cells injected into the donor pig undergo differentiation and maturation into regulatory cells, suppressor cells, select B cells, and antibodies in the pig donor while in utero, wherein the differentiated and matured cells mediate specific tolerance when these cells are given back to the recipient in conjunction with the pig organ from the same pig. Another potential benefit of injecting human cells and factors into the fetal pig, is the potential for accommodation of pig endothelial cells to human proteins which may reduce the potential metabolic incompatibilities associated with protein/ligand differences between species.

26. The method of any one of paragraphs 1 to 25, wherein the human cells removed, expanded, and purified after the donor piglet is delivered comprise (i) foxP3+CD4+CD3+CD25+ natural T regulatory cells, (ii) adaptive CD4+CD49b+LAG3+CD226+ regulatory Tr1 cells, (iii) CD4+CD25-CD69+foxP3-LAP+(TGF beta producing type 3 helper T cells (TH 3), and (iv) dendritic cells expressing PD-L1.

27. A donor pig useful for xenotransplantation of an organ that is personalized to a specific organ recipient and that does not require chronic immunosuppression following xenotransplantation,
wherein the donor pig is a genetically modified donor pig that was delivered from a genetically modified parent pig, wherein the genetic modifications of the donor pig are the same as the genetic modifications of the parent pig, wherein the genetic modifications are limited to three gene knockouts and, optionally, three human transgenes, and wherein the donor pig is the same sex and compatible blood type as the organ recipient,
wherein cells and factors of the organ recipient were transferred to the GM donor pig while the donor pig was a donor pig fetus in utero in the parent pig, wherein, prior to transferring, the cells and factors of the organ recipient (i) were collected from blood and bone marrow of the organ recipient and (ii) were partially depleted of CD4+ and CD8+ T cells.

28. The donor pig of paragraph 27, wherein the donor pig is a species selected from Sus scrofa domesticas, Sus ahoenobarbus, Sus barbatus, Sus bucculentus, Sus cebifrons, Sus celebensis, Sus heureni, Sus oliveri, Sus philippensi, and Sus verrucosus.

29. The donor pig of any one of paragraphs 27 or 28, wherein the pig is a Sus scrofa domesticas pig.

30. The donor pig of paragraph 29, wherein the pig is a Yucatan pig.

31. An organ from a GM donor pig useful for xenotransplantation that is personalized to a specific organ recipient and that does not require chronic immunosuppression following xenotransplantation,
wherein the donor pig is a genetically modified donor pig that was delivered from a genetically modified parent pig, wherein the genetic modifications of the donor pig are the same as the genetic modifications of the parent pig, wherein the genetic modifications are limited to three gene knockouts and, optionally, three human transgenes, and wherein the donor pig is the same sex and compatible blood group as the organ recipient,
wherein cells and factors of the organ recipient were transferred to the GM donor pig while the donor pig was a donor pig fetus in utero in the parent pig, wherein, prior to transferring, the cells of the organ recipient (i) were collected from blood and bone marrow of the organ recipient and (ii) were partially depleted of CD4+ and CD8+ T cells.

32. The organ of paragraph 31, wherein the organ from the GM donor pig comprises all or part of a heart, bone, liver, lung, kidney, pancreas, eye, or intestine.

33. A method of treating a subject having a disease or disorder, comprising transplanting into the subject the organ of any one of paragraphs 31 or 32,
wherein the transplanting is effective to treat or prevent the disease or disorder in the subject.

34. The method of paragraph 33, wherein the disease or disorder is selected from the group consisting of heart diseases, kidney diseases, liver diseases, lung diseases, eye diseases, pancreatic diseases, and congenital diseases of one or more organs.

35. Cells and factors from a GM donor pig useful for supporting xenotransplantation of an organ of the donor pig that is personalized to a specific organ recipient and that does not require chronic immunosuppression following xenotransplantation,
wherein the donor pig is a genetically modified donor pig that was delivered from a genetically modified parent pig, wherein the genetic modifications of the donor pig are the same as the genetic modifications of the parent pig, wherein the genetic modifications are limited to three gene knockouts and, optionally, three human transgenes, and wherein the donor pig is the same sex and compatible blood group as the organ recipient,
wherein cells and factors of the organ recipient were transferred to the GM donor pig while the donor pig was a donor pig fetus in utero in the parent pig, wherein, prior to transferring, the cells and factors of the organ recipient (i) were collected from blood and bone marrow of the organ recipient and (ii) were partially depleted of CD4+ and CD8+ T cells,
wherein the cells and factors of the GM donor pig (i) were collected from blood, bone marrow, cord blood, spleen, and lymph nodes of the donor pig, (ii) were enriched for organ recipient cells and depleted of donor pig cells, (iii) were enriched for organ recipient foxP3+CD4+CD3+CD25+ natural T regulatory cells and expanded, (iv) were enriched for organ recipient adaptive CD4+CD49b+LAG3+CD226+ regulatory Tr1 cells and expanded, and (v) were enriched for organ recipient CD4+CD25-CD69+foxP3-LAP+(TGFβ-producing type 3 helper T cells) and expanded; and (vi) were enriched for organ recipient PD-L1 human dendritic cells and expanded.

36. The cells and factors of paragraph 35, wherein the cells comprise one or more cell types selected from the group consisting of skin and bone cells (fibroblasts, osteoblasts, osteocytes, osteoclasts, osteoprogenitor cells), cardiac cells (cardiac fibroblasts (CFs), cardiomyocytes, smooth muscle cells (SMCs), and endothelial cells (ECs)), blood cells (red blood cells, white blood cells), liver cells ((hepatocytes (HCs), hepatic stellate cells (HSCs), kupffer cells (KCs), and liver sinusoidal endothelial cells (LSECs)), kidney cells (glomerular basement membrane/glomerular endothelial cells, macula densa cells, mesangial cells, podocytes, tubule epithelial cells), cells of the pancreas (islet cells, including alpha, beta and delta cells), and stem cells.

37. A genetically modified pig, wherein the genetic modifications are limited to three gene knockouts and, optionally, three human transgenes,
wherein the gene knockouts are Glycoprotein Alpha-Galactosyltransferase 1 (GGTA1), Cytidine Monophospho-N-Acetylneuraminic Acid Hydroxylase (CMAH), and Beta-1,4-N-Acetyl-Galactosaminyltransferase 2 (B4GALNT2),
wherein the human transgenes are human Thrombomodulin (hTBM), human Membrane Cofactor Protein (hCD46), and human Decay Accelerating Factor For Complement (hCD55).

38. The genetically modified pig of paragraph 37, wherein the donor pig is a species selected from Sus scrofa domesticas, Sus ahoenobarbus, Sus barbatus, Sus bucculentus, Sus cebifrons, Sus celebensis, Sus heureni, Sus oliveri, Sus philippensi, and Sus verrucosus.

39. The genetically modified pig of any one of paragraphs 37 or 38, wherein the pig is a Sus scrofa domesticas pig.

40. The genetically modified pig of paragraph 39, wherein the pig is s Yucatan pig.

41. A method of producing and using genetically modified (GM) animal xenotransplantation materials that are personalized to a specific xenotransplant recipient and that do not require chronic immunosuppression following xenotransplantation, the method comprising:
(a) transferring cells and factors of the xenotransplant recipient into a GM donor animal fetus in utero, wherein the donor animal is a non-human mammal, wherein the xenotransplant recipient is a human or a non-human mammal of a different species than the donor animal,
   wherein, prior to transferring, the cells and factors of the xenotransplant recipient (i) are collected from blood and bone marrow of the xenotransplant recipient and (ii) are partially depleted of CD4+ and CD8+ T cells,
   wherein the transferring is accomplished by injecting the partially depleted cells and factors of the xenotransplant recipient into the abdominal cavity of the GM donor animal fetus, wherein the donor animal fetus is a genetically modified donor animal fetus being gestated in a genetically modified parent animal, wherein the genetic modifications of the donor animal fetus are the same as the genetic modifications of the parent animal, wherein the genetic modifications are limited to three gene knockouts and, optionally, three transgenes from the species of the xenotransplant recipient, and wherein the GM donor animal fetus is the same sex and compatible blood group as the xenotransplant recipient;
(b) transferring, after completion of gestation of the GM donor animal fetus and delivery and some development of the resultant GM donor animal, the xenotransplant recipient's cells and factors from the GM donor animal back into the xenotransplant recipient,
   wherein, prior to transferring, the cells and factors of the GM donor animal (i) are collected from one or more of blood, bone marrow, spleen, cord blood and lymph nodes of the GM donor animal, (ii) are depleted of donor animal cells, and (iii) are subject to enrichment, expansion, and/or purification for the xenotransplant recipient's cells selected from the group consisting of foxP3+CD4+CD3+CD25+ natural T regulatory cells, adaptive CD4+CD49b+LAG3+CD226+ regulatory Tr1 cells, CD4+CD25-CD69+foxP3-LAP+(TGFβ-producing type 3 helper T cells), and xenotransplant recipient PD-L1 dendritic cells,
   wherein, 5 to 9 days prior to transferring, the xenotransplant recipient is administered Cyclophosphamide⁺/− Fludarabine and
(c) transplanting, 5 to 9 days following the transferring of the xenotransplant recipient's cells and factors from the GM donor animal back into the xenotransplant recipient, an xenotransplant organ from the same GM donor animal is placed into the xenotransplant recipient.

42. The method of paragraph 41, further comprising administering one or more anti-inflammatory drugs to the xenotransplant recipient post-xenotransplant.

43. The method of paragraph 42, wherein the one or more anti-inflammatory drugs are administered to the xenotransplant recipient for 6-12 months.

44. The method of any one of paragraphs 41 to 43, wherein the xenotransplant recipient is a subject having been diagnosed as having a disease or disorder selected from the group consisting of heart diseases, kidney diseases, liver diseases, lung diseases, eye diseases, pancreatic diseases, and congenital diseases of one or more organs.

45. The method of any one of paragraphs 41 to 44, wherein the xenotransplant from the GM donor animal comprises all or part of a heart, bone, liver, lung, kidney, pancreas, intestine, or eye.

46. The method of any one of paragraphs 41 to 45, wherein the xenotransplant from the GM donor animal comprises one or more cell types selected from the group consisting of skin and bone cells (fibroblasts, osteoblasts, osteocytes, osteoclasts, osteoprogenitor cells), cardiac cells (fibroblasts (CFs), cardiomyocytes, smooth muscle cells (SMCs), and endothelial cells (ECs)), blood cells (red blood cells, white blood cells), liver cells (hepatocytes (HCs), hepatic stellate cells (HSCs), kupffer cells (KCs), and liver sinusoidal endothelial cells (LSECs)), kidney cells (glomerular basement membrane/glomerular endothelial cells, macula densa cells, mesangial cells, podocytes, tubule epithelial cells), cells of the pancreas (islet cells, including alpha, beta and delta cells), and stem cells.

47. The method of any one of paragraphs 41 to 46, wherein the GM donor animal is at the same site as the recipient during the transplanting in step (c).

48. The method of any one of paragraphs 41 to 47, wherein the GM donor animal and the recipient are not at the same site during the transplanting in step (c), and wherein the method further comprises one or more steps for storing and transporting the xenotransplant from the donor site to the recipient site, optionally via an xenotransplant pig organ storage bank.

49. The method of any one of paragraphs 41 to 48, wherein the donor animal is a species selected from Sus scrofa domesticas, Sus ahoenobarbus, Sus barbatus, Sus bucculentus, Sus cebifrons, Sus celebensis, Sus heureni, Sus oliveri, Sus philippensis, and Sus verrucosus.

50. The method of any one of paragraphs 41 to 49, wherein the animal is a Sus scrofa domesticas pig.

51. The method of paragraph 10, wherein the pig is a Yucatan pig.

52. The method of any one of paragraphs 41 to 51, wherein the cells of the xenotransplant recipient in step (a) are selected from the group consisting of lymphocytes and stem cells.

53. The method of any one of paragraphs 41 to 52, wherein the animal xenotransplant after transplanting in the recipient grows less than 5%, 10%, 15%, or 15% in volume.

54. The method of any one of paragraphs 41 to 53, wherein the donor animal is chosen so that the animal xenotransplant will not continue to grow in the recipient after transplantation.

55. The method of any one of paragraphs 41 to 54, wherein the GM animal comes from a GM animal population was expanded with minimal in-breeding.

56. The method of any one of paragraphs 41 to 55, wherein the genetic modifications to the donor animals are limited to fewer that seven genetic modifications.

57. The method of any one of paragraphs 41 to 56, wherein the genetic modifications are made using Somatic Cell Nuclear Transfer (SCNT), CRISPR-Cas, or other methods.

58. The method of any one of paragraphs 41 to 57, wherein the genetic modifications in the animal include knockouts of 3 animal genes and the addition of 3 transgenes of the species of the xenotransplant recipient.

59. The method of any one of paragraphs 41 to 58, wherein Porcine Endogenous Retrovirus-C (PERV-C) is eliminated in the GM animal.

60. The method of any one of paragraphs 41 to 59, wherein the GM animals are raised in a pathogen-free environment, are tested for infectious agents of concern, and are cleared of all viruses of concern.

61. The method of any one of paragraphs 41 to 60, wherein the recipient is more personally matched to the GM animal, wherein the animal organs in the GM animal are repopulated with the recipient's cells to make the animal organs more like those of the xenotransplant recipient.

62. The method of any one of paragraphs 41 to 61, wherein the removal of the recipient's lymphocytes comprises the removal of hematopoietic and mesenchymal stem cells and other factors from the recipient's blood and bone marrow.

63. The method of any one of paragraphs 41 to 62, wherein the injection of the recipient's lymphocytes comprises the injection of the recipient's hematopoietic and mesenchymal stem cells and other factors into the donor animal in utero.

64. The method of any one of paragraphs 41 to 63, wherein the recipient's lymphocytes, hematopoietic and mesenchymal stem cells injected into the donor animal in utero are tracked.

65. The method of any one of paragraphs 41 to 64, wherein the recipient's lymphocytes and stems cells injected into the donor animal undergo differentiation and maturation into regulatory cells, suppressor cells, select B cells, and antibodies in the animal donor while in utero, wherein the differentiated and matured cells mediate specific tolerance when these cells are given back to the recipient in conjunction with the animal organ xenotransplant from the same animal.

66. The method of any one of paragraphs 41 to 65, wherein the mammalian cells removed, expanded, and purified after the donor animal is delivered comprise (i) foxP3+CD4+CD3+CD25+ natural T regulatory cells, (ii) adaptive CD4+CD49b+LAG3+CD226+ regulatory Tr1 cells, (iii) CD4+CD25-CD69+foxP3-LAP+(TGF beta producing type 3 helper T cells (TH 3), and (iv) dendritic cells expressing PD-L1.

67. A donor animal useful for xenotransplantation of an xenotransplant that is personalized to a specific xenotransplant recipient and that does not require chronic immunosuppression following xenotransplantation, wherein the donor animal is a non-human mammal, wherein the xenotransplant recipient is a human or a non-human mammal of a different species than the donor animal,
wherein the donor animal is a genetically modified donor animal that was delivered from a genetically modified parent animal, wherein the genetic modifications of the donor animal are the same as the genetic modifications of the parent animal, wherein the genetic modifications are limited to three gene knockouts and, optionally, three mammalian transgenes, and wherein the donor animal is the same sex and compatible blood group as the xenotransplant recipient,
wherein cells and factors of the xenotransplant recipient were transferred to the GM donor animal while the donor animal was a donor animal fetus in utero in the parent animal, wherein, prior to transferring, the cells and factors of the xenotransplant recipient (i) were collected from blood and bone marrow of the xenotransplant recipient and (ii) were partially depleted of CD4+ and CD8+ T cells.

68. The donor animal of paragraph 67, wherein the donor animal is a species selected from Sus scrofa domesticas, Sus ahoenobarbus, Sus barbatus, Sus bucculentus, Sus cebifrons, Sus celebensis, Sus heureni, Sus oliveri, Sus philippensi, and Sus verrucosus.

69. The donor animal of any one of paragraphs 67 or 68, wherein the pig is a Sus scrofa domesticas pig.

70. The donor animal of paragraph 69, wherein the animal is a Yucatan pig.

71. An xenotransplant from a GM donor animal useful for xenotransplantation that is personalized to a specific xenotransplant recipient and that does not require chronic immunosuppression following xenotransplantation, wherein the donor animal is a non-human mammal, wherein the xenotransplant recipient is a human or a non-human mammal of a different species than the donor animal,
wherein the donor animal is a genetically modified donor animal that was delivered from a genetically modified parent animal, wherein the genetic modifications of the donor animal are the same as the genetic modifications of the parent animal, wherein the genetic modifications are limited to three gene knockouts and, optionally, three mammalian transgenes, and wherein the donor animal is the same sex and compatible blood group as the xenotransplant recipient,
wherein cells and factors of the xenotransplant recipient were transferred to the GM donor animal while the donor animal was a donor animal fetus in utero in the parent animal, wherein, prior to transferring, the cells of the xenotransplant recipient (i) were collected from blood and bone marrow of the xenotransplant recipient and (ii) were partially depleted of CD4+ and CD8+ T cells.

72. The xenotransplant of paragraph 71, wherein the xenotransplant from the GM donor animal comprises all or part of a heart, bone, liver, lung, kidney, pancreas, intestine, or eye.

73. A method of treating a subject having a disease or disorder, comprising transplanting into the subject the xenotransplant of any one of paragraphs 71 or 72,
wherein the transplanting is effective to treat or prevent the disease or disorder in the subject.

74. The method of paragraph 73, wherein the disease or disorder is selected from the group consisting of heart diseases, kidney diseases, liver diseases, lung diseases, eye diseases, pancreatic diseases, and congenital diseases of one or more organs.

75. Cells and factors from a GM donor animal useful for supporting xenotransplantation of the donor animal that is personalized to a specific xenotransplant recipient and that does not require chronic immunosuppression following xenotransplantation, wherein the donor animal is a non-human mammal, wherein the xenotransplant recipient is a human or a non-human mammal of a different species than the donor animal,
wherein the donor animal is a genetically modified donor animal that was delivered from a genetically modified parent animal, wherein the genetic modifications of the donor animal are the same as the genetic modifications of the parent animal, wherein the genetic modifications are limited to three gene knockouts and, optionally, three mammalian transgenes, and wherein the donor animal is the same sex and compatible blood group as the xenotransplant recipient,
wherein cells and factors of the xenotransplant recipient were transferred to the GM donor animal while the donor animal was a donor animal fetus in utero in the parent animal, wherein, prior to transferring, the cells and factors of the xenotransplant recipient (i) were collected from blood and bone marrow of the xenotransplant recipient and (ii) were partially depleted of CD4+ and CD8+ T cells,
wherein the cells and factors of the GM donor animal (i) were collected from blood, bone marrow, cord blood, spleen, and lymph nodes of the donor animal, (ii) were enriched for xenotransplant recipient cells and depleted of donor animal cells, (iii) were enriched for xenotransplant recipient foxP3+CD4+CD3+CD25+ natural T regulatory cells and expanded, (iv) were enriched for xenotransplant recipient adaptive CD4+CD49b+LAG3+CD226+ regulatory Tr1 cells and expanded, and (v) were enriched for xenotransplant recipient CD4+CD25-CD69+foxP3-LAP+(TGFβ-producing type 3 helper T cells) and expanded; and/or (vi) were enriched for xenotransplant recipient PD-L1 dendritic cells and expanded.

76. The cells and factors of paragraph 75, wherein the cells comprise one or more cell types selected from the group consisting of skin and bone cells (fibroblasts, osteoblasts, osteocytes, osteoclasts, osteoprogenitor cells), cardiac cells (cardiac fibroblasts (CFs), cardiomyocytes, smooth muscle cells (SMCs), and endothelial cells (ECs)), blood cells (red blood cells, white blood cells), liver cells ((hepatocytes (HCs), hepatic stellate cells (HSCs), Kupffer cells (KCs), and liver sinusoidal endothelial cells (LSECs)), kidney cells (glomerular basement membrane/glomerular endothelial cells, macula densa cells, mesangial cells, podocytes, tubule epithelial cells), cells of the pancreas (islet cells, including alpha, beta and delta cells), and stem cells.

77. A genetically modified animal, wherein the genetic modifications are limited to three gene knockouts and, optionally, three mammalian transgenes, wherein the donor animal is a non-human mammal, wherein the mammalian transgenes are human or from a non-human mammal of a different species than the donor animal,
wherein the gene knockouts are Glycoprotein Alpha-Galactosyltransferase 1 (GGTA1), Cytidine Monophospho-N-Acetylneuraminic Acid Hydroxylase (CMAH), and Beta-1,4-N-Acetyl-Galactosaminyltransferase 2 (B4GALNT2),
wherein the mammalian transgenes are mammalian Thrombomodulin (hTBM), mammalian Membrane Cofactor Protein (hCD46), and mammalian Decay Accelerating Factor for Complement (hCD55).

78. The genetically modified animal of paragraph 77, wherein the donor animal is a species selected from Sus scrofa domesticas, Sus ahoenobarbus, Sus barbatus, Sus bucculentus, Sus cebifrons, Sus celebensis, Sus heureni, Sus oliveri, Sus philippensi, and Sus verrucosus.

79. The genetically modified animal of any one of paragraphs 77 or 78, wherein the animal is a Sus scrofa domesticas pig.

80. The genetically modified animal of paragraph 79, wherein the animal is s Yucatan pig.

We claim:

1. A method of producing and using genetically modified (GM) pig organs for xenotransplantation that are personalized to a specific organ recipient and that do not require chronic immunosuppression following xenotransplantation, the method comprising:
(a) transferring cells and factors of the organ recipient into a GM donor pig fetus in utero,
wherein, prior to transferring, the cells and factors of the organ recipient (i) are collected from blood and bone marrow of the organ recipient and (ii) are partially depleted of CD4+ and CD8+ T cells,
wherein the transferring is accomplished by injecting the partially depleted cells and factors of the organ recipient into the abdominal cavity of the GM donor pig fetus, wherein the donor pig fetus is a genetically modified donor pig fetus being gestated in a genetically modified parent pig, wherein the genetic modifications of the donor pig fetus are the same as the genetic modifications of the parent pig, wherein the genetic modifications are limited to three gene knockouts and, optionally, three human transgenes, and wherein the GM donor pig fetus is the same sex and compatible blood group as the organ recipient;
(b) transferring, after completion of gestation of the GM donor pig fetus and delivery and some development of the resultant GM donor pig, the organ recipient's cells and factors from the GM donor pig back into the organ recipient,
wherein, prior to transferring, the cells and factors of the GM donor pig (i) are collected from one or more of blood, bone marrow, spleen, cord blood and lymph nodes of the GM donor pig, (ii) are depleted of donor pig cells, and (iii) are subject to enrichment, expansion, and/or purification for the organ recipient's cells selected from the group consisting of foxP3+CD4+CD3+CD25+ natural T regulatory cells, adaptive CD4+CD49b+LAG3+CD226+ regulatory Tr1 cells, CD4+CD25-CD69+foxP3-LAP+(TGFβ-producing type 3 helper T cells), and organ recipient PD-L1 human dendritic cells,
wherein, 5 to 9 days prior to transferring, the organ recipient is administered Cyclophosphamide +/− Fludarabine and
(c) transplanting, 5 to 9 days following the transferring of the organ recipient's cells and factors from the GM donor pig back into the organ recipient, an organ from the same GM donor pig into the organ recipient.

2. The method of claim 1, further comprising administering one or more anti-inflammatory drugs to the organ recipient post-xenotransplant.

3. The method of claim 2, wherein the one or more anti-inflammatory drugs are administered to the organ recipient for 6-12 months.

4. The method of claim 1, wherein the organ recipient is a subject having been diagnosed as having a disease or disorder selected from the group consisting of heart diseases, kidney diseases, liver diseases, lung diseases, eye diseases, pancreatic diseases, and congenital diseases of one or more organs.

5. The method of claim 1, wherein the organ from the GM donor pig comprises all or part of a heart, bone, liver, lung, kidney, pancreas, eye, or intestine.

6. The method of claim 1, wherein the organ from the GM donor pig comprises one or more cell types selected from the group consisting of skin and bone cells (e.g., fibroblasts, osteoblasts, osteocytes, osteoclasts, osteoprogenitor cells), cardiac cells (e.g., fibroblasts (CFs), cardiomyocytes, smooth muscle cells (SMCs), and endothelial cells (ECs)), blood cells (e.g., red blood cells, white blood cells), liver cells (e.g., hepatocytes (HCs), hepatic stellate cells (HSCs), Kupffer cells (KCs), and liver sinusoidal endothelial cells (LSECs)), kidney cells (e.g., glomerular basement membrane/glomerular endothelial cells, macula densa cells, mesangial cells, podocytes, tubule epithelial cells), cells of the pancreas (e.g., islet cells, including alpha, beta and delta cells), and stem cells.

7. The method of claim 1, wherein the GM donor pig is at the same site as the recipient during the transplanting in step (c).

8. The method of claim 1, wherein the GM donor pig and the recipient are not at the same site during the transplanting in step (c), and
wherein the method further comprises one or more steps for storing and transporting the organ from the donor site to the recipient site, optionally via a pig organ storage bank.

9. The method of claim 1, wherein the donor pig is a species selected from Sus scrofa domesticas, Sus ahoenobarbus, Sus barbatus, Sus bucculentus, Sus cebifrons, Sus celebensis, Sus heureni, Sus oliveri, Sus philippensis, and Sus verrucosus.

10. The method of claim 1, wherein the pig is a Sus scrofa domesticas pig.

11. The method of claim 10, wherein the pig is a Yucatan pig.

12. The method of claim 1, wherein the cells of the organ recipient in step (a) are selected from the group consisting of lymphocytes and stem cells.

13. The method of claim 1, wherein the pig organ after transplanting in the recipient grows less than 5%, 10%, 15%, or 15% in volume.

14. The method of claim 1, wherein the donor pig is chosen so that the pig organ will not continue to grow in the recipient after transplantation.

15. The method of claim 1, wherein the GM pig comes from a GM pig population was expanded with minimal in-breeding.

16. The method of claim 1, wherein the genetic modifications to the donor pigs are limited to fewer that seven genetic modifications.

17. The method of claim 1, wherein the genetic modifications are made using Somatic Cell Nuclear Transfer (SCNT).

18. The method of claim 1, wherein the genetic modifications in the pig include knockouts of 3 pig genes and the addition of 3 human transgenes.

19. The method of claim 1, wherein Porcine Endogenous Retrovirus-C (PERV-C) is eliminated in the pig organ.

20. The method of claim 1, wherein the GM pigs are raised in a pathogen-free environment, are tested for infectious agents of concern, and are cleared of all viruses of concern.

21. The method of claim 1, wherein the recipient has a more personally matched GM pig compared to equivalent GM pigs raised in the absence of step (a) and/or step (b), wherein the pig organ in the GM pig is repopulated with the recipient's cells to make the pig organ more humanized.

22. The method of claim 1, wherein the removal of the recipient's lymphocytes comprises the removal of hematopoietic and mesenchymal stem cells and other factors from the recipient's blood and bone marrow.

23. The method of claim 1, wherein the injection of the recipient's lymphocytes comprises the injection of the recipient's hematopoietic and mesenchymal stem cells and other factors into the donor pig in utero.

24. The method of claim 1, wherein the recipient's lymphocytes, hematopoietic and mesenchymal stem cells injected into the donor pig in utero are tracked.

25. The method of claim 1, wherein the recipient's lymphocytes and stems cells injected into the donor pig undergo differentiation and maturation into regulatory cells, suppressor cells, select B cells, and antibodies in the pig donor while in utero, wherein the differentiated and matured cells mediate specific tolerance when these cells are given back to the recipient in conjunction with the pig organ from the same pig.

26. The method of any one of the claim 1, wherein a potential benefit of injecting human cells and factors into the fetal pig, is the potential accommodation of the pig endothelial cells to human proteins and may reduce the potential metabolic incompatibilities associated with protein/ligand differences between species.

27. The method of claim 1, wherein the human cells removed, expanded, and purified after the donor piglet is delivered comprise (i) foxP3+CD4+CD3+CD25+ natural T regulatory cells, (ii) adaptive CD4+CD49b+LAG3+CD226+ regulatory Tr1 cells, (iii) CD4+CD25-CD69+foxP3-LAP+(TGF beta producing type 3 helper T cells (TH 3), and (iv) dendritic cells expressing PD-L1.

28. A donor pig useful for xenotransplantation of an organ that is personalized to a specific organ recipient and that does not require chronic immunosuppression following xenotransplantation,
wherein the donor pig is a genetically modified donor pig that was delivered from a genetically modified parent pig, wherein the genetic modifications of the donor pig are the same as the genetic modifications of the parent pig, wherein the genetic modifications are limited to three gene knockouts and, optionally, three human transgenes, and wherein the donor pig is the same sex and compatible blood type as the organ recipient,
wherein cells and factors of the organ recipient were transferred to the GM donor pig while the donor pig was a donor pig fetus in utero in the parent pig, wherein, prior to transferring, the cells and factors of the organ recipient (i) were collected from blood and bone marrow of the organ recipient and (ii) were partially depleted of CD4+ and CD8+ T cells.

29. The donor pig of claim 28, wherein the donor pig is a species selected from Sus scrofa domesticas, Sus ahoenobarbus, Sus barbatus, Sus bucculentus, Sus cebifrons, Sus celebensis, Sus heureni, Sus oliveri, Sus philippensi, and Sus verrucosus.

30. The donor pig of claim 28, wherein the pig is a Sus scrofa domesticas pig.

31. The donor pig of claim 30, wherein the pig is a Yucatan pig.

32. An organ from a GM donor pig useful for xenotransplantation that is personalized to a specific organ recipient and that does not require chronic immunosuppression following xenotransplantation,
wherein the donor pig is a genetically modified donor pig that was delivered from a genetically modified parent pig, wherein the genetic modifications of the donor pig are the same as the genetic modifications of the parent pig, wherein the genetic modifications are limited to three gene knockouts and, optionally, three human transgenes, and wherein the donor pig is the same sex and compatible blood group as the organ recipient,
wherein cells and factors of the organ recipient were transferred to the GM donor pig while the donor pig was a donor pig fetus in utero in the parent pig, wherein, prior to transferring, the cells of the organ recipient (i) were collected from blood and bone marrow of the organ recipient and (ii) were partially depleted of CD4+ and CD8+ T cells.

33. The organ of claim 32, wherein the organ from the GM donor pig comprises all or part of a heart, bone, liver, lung, kidney, pancreas, eye, or intestine.

34. A method of treating a subject having a disease or disorder, comprising transplanting into the subject the organ of claim 32,
wherein the transplanting is effective to treat or prevent the disease or disorder in the subject.

35. The method of claim 34, wherein the disease or disorder is selected from the group consisting of heart diseases, kidney diseases, liver diseases, lung diseases, eye diseases, pancreatic diseases, and congenital diseases of one or more organs.

36. Cells and factors from a GM donor pig useful for supporting xenotransplantation of an organ of the donor pig that is personalized to a specific organ recipient and that does not require chronic immunosuppression following xenotransplantation,
wherein the donor pig is a genetically modified donor pig that was delivered from a genetically modified parent pig, wherein the genetic modifications of the donor pig are the same as the genetic modifications of the parent pig, wherein the genetic modifications are limited to three gene knockouts and, optionally, three human transgenes, and wherein the donor pig is the same sex and compatible blood group as the organ recipient,
wherein cells and factors of the organ recipient were transferred to the GM donor pig while the donor pig was a donor pig fetus in utero in the parent pig, wherein, prior to transferring, the cells and factors of the organ recipient (i) were collected from blood and bone marrow of the organ recipient and (ii) were partially depleted of CD4+ and CD8+ T cells,
wherein the cells and factors of the GM donor pig (i) were collected from blood, bone marrow, cord blood, spleen, and lymph nodes of the donor pig, (ii) were enriched for organ recipient cells and depleted of donor pig cells, (iii) were enriched for organ recipient foxP3+CD4+CD3+CD25+ natural T regulatory cells and expanded, (iv) were enriched for organ recipient adaptive CD4+CD49b+LAG3+CD226+ regulatory Tr1 cells and expanded, and (v) were enriched for organ recipient CD4+CD25-CD69+foxP3-LAP+(TGFβ-producing type 3 helper T cells) and expanded; and/or (vi) were enriched for organ recipient PD-L1 human dendritic cells and expanded.

37. The cells and factors of claim 36, wherein the cells comprise one or more cell types selected from the group consisting of skin and bone cells (e.g., fibroblasts, osteoblasts, osteocytes, osteoclasts, osteoprogenitor cells), cardiac cells (e.g., cardiac fibroblasts (CFs), cardiomyocytes, smooth muscle cells (SMCs), and endothelial cells (ECs)), blood cells (e.g., red blood cells, white blood cells), liver cells (e.g., hepatocytes (HCs), hepatic stellate cells (HSCs), Kupffer cells (KCs), and liver sinusoidal endothelial cells (LSECs)), kidney cells (e.g., glomerular basement membrane/glomerular endothelial cells, macula densa cells, mesangial cells, podocytes, tubule epithelial cells), cells of the pancreas (e.g., islet cells, including alpha, beta and delta cells), and stem cells.

38. A genetically modified pig, wherein the genetic modifications are limited to three gene knockouts and, optionally, three human transgenes,
wherein the gene knockouts are Glycoprotein Alpha-Galactosyltransferase 1 (GGTA1), Cytidine Monophospho-N-Acetylneuraminic Acid Hydroxylase (CMAH), and Beta-1,4-N-Acetyl-Galactosaminyltransferase 2 (B4GALNT2),
wherein the human transgenes are human Thrombomodulin (hTBM), human Membrane Cofactor Protein (hCD46), and human Decay Accelerating Factor for Complement (hCD55).

39. The genetically modified pig of claim 38, wherein the donor pig is a species selected from Sus scrofa domesticas, Sus ahoenobarbus, Sus barbatus, Sus bucculentus, Sus cebifrons, Sus celebensis, Sus heureni, Sus oliveri, Sus philippensi, and Sus verrucosus.

40. The genetically modified pig of claim 38, wherein the pig is a Sus scrofa domesticas pig.

41. The genetically modified pig of claim 40, wherein the pig is a Yucatan pig.
